(19) United States
(12) Patent Application Publication
(10) Pub. No.: US 2024/0138732 A1
(43) Pub. Date: May 2, 2024

(54) CAPILLARY BLOOD SAMPLING DEVICE AND METHOD OF USE

(71) Applicant: Preci Health SA, Neuchâtel (CH)

(72) Inventors: Alain JACCARD, Ste-Croix (CH); Lucien VOUILLAMOZ, Feusisberg (CH); Philippe MARGAIRAZ, La Chaux-de-Fonds (CH); Shay ZYMAN, Pardes Hanna (IL)

(73) Assignee: Preci Health SA, Neuchâtel (CH)

(21) Appl. No.: 18/380,284

(22) Filed: Oct. 16, 2023

Related U.S. Application Data
(63) Continuation-in-part of application No. 18/022,772, filed on Feb. 23, 2023.
(60) Provisional application No. 63/468,803, filed on May 25, 2023.
(60) Provisional application No. 63/523,675, filed on Jun. 28, 2023.
(60) Provisional application No. 63/379,741, filed on Oct. 15, 2022.
(60) Provisional application No. 63/069,112, filed on Aug. 23, 2020.
(60) Provisional application No. 63/142,756, filed on Jan. 28, 2021.
(60) Provisional application No. 63/150,113, filed on Feb. 17, 2021.
(60) Provisional application No. 63/153,088, filed on Feb. 24, 2021.

(30) Foreign Application Priority Data
Mar. 31, 2021 (WO) PCT/IB2021/000187

(51) Int. Cl. A61B 5/15

(52) U.S. Cl. CPC A61B 5/150022; A61B 5/150076; A61B 5/150221; A61B 5/150343

(57) ABSTRACT

A disposable fluid sampling device including a body fluid sampling means, optionally body fluid sampling means for auto-sampling; and optionally, analysis means using one or more droplet(s) of sampled fluid to analyze the fluid; and an interface for at least one sample tube or medical analysis tube, the at least one sample tube or medical analysis tube adapted to be filled with a sample of fluid for analysis in a point of care or medical lab, wherein the device includes an interface for and at least one vacuum tube or vacuum source, the vacuum tube or vacuum source adapted to provide suction necessary to fill the at least one sample tube with the fluid.

### COPYRIGHT & LEGAL NOTICE

A portion of the disclosure of this patent document contains material which is subject to copyright protection. The Applicant has no objection to the facsimile reproduction by anyone of the patent document or the patent disclosure as it appears in the Patent and Trademark Office patent file or records, but otherwise reserves all copyright rights whatsoever. Further, no references to third party patents or articles made herein is to be construed as an admission that the present invention is not entitled to antedate such material by virtue of prior invention.

### FIELD OF THE INVENTION

This invention relates to devices allowing for the sampling of capillary blood. In many cases it is preferable to avoid venipuncture to sample blood, be it because the patient's veins are too fragile, or for comfort reasons, or because trained personnel for phlebotomy are not available in sufficient numbers.

### BACKGROUND OF THE INVENTION

The sampling of capillary blood is often preferable to venous blood extraction because a patient's veins may be fragile or for comfort reasons, or because trained personnel for phlebotomy is not available in sufficient numbers.

What is needed is a capillary fluid sampling device adapted to be used safely and able to provide the necessary blood samples for analysis devices or analysis systems at the point of care or at a medical laboratory. What is needed is a capillary blood collection device that can, using a single laceration or puncture system at a single location, collect one or ideally successively multiple blood samples of at least 500 μl per sample per tube, in one or ideally in multiple standard tubes for the purpose of performing blood tests, using vacuum from a vacuum tube or several vacuum tubes or from an external vacuum source.

### SUMMARY OF THE INVENTION

A disposable body fluid sampling device provides a user with the ability to sample capillary blood and fill one or more sample tubes for analyses. The disposable body fluid sampling device may include: a body fluid reservoir for containing the sampled body fluid and a fluid extraction mechanism including at least one conduit or hollow needle connected to a vacuum reservoir or external vacuum source. When connecting the vacuum reservoir or external vacuum source to the body fluid reservoir, a seal of the vacuum reservoir or external vacuum source is adapted to be broken, pierced or opened so as to cause the sucking of blood into the body fluid reservoir. The sampling device of the invention permits collecting capillary blood samples without the intervention of medically trained personnel, in particular in the absence of personnel trained in phlebotomy. The sampling device enables the user to: (a) sample a body fluid, or optionally carry out auto-sampling; (b) optionally using one or more droplet(s) of the sampled body fluid, to immediately analyze the body fluid; and (c) provide one or more medical analysis tubes filled with the sampled body fluid for analysis at a point of care or in a medical lab. The sampling device may include a vacuum tube or external vacuum source and an interface therefor. The vacuum tube or external vacuum source provides the suction necessary to draw the body fluid from the user/patient and to fill the body fluid reservoir with the body fluid.

The task is solved by a fluid sampling device adapted to take a sample of a body fluid, preferably the sample of the body fluid formed from one or more droplets of the body fluid for optional immediate analysis and from larger quantities of body fluid (0.5 to 1.5 ml per sample tube) similar to those collected by phlebotomy for further analysis at a point of care or in a medical lab, the disposable fluid sampling device optionally adapted for auto-sampling, wherein the disposable fluid sampling device includes a vacuum interface for interfacing a first vacuum source and a second vacuum source, the vacuum sources interconnected to the at least one receptacle for providing suction necessary to fill the at least one receptacle with the body fluid. Preferably the body fluid is blood. The fluid sampling device is optionally disposable.

In the present disclosure the description of the body fluid sampling device is described in particular for the case of sampling capillary blood, but the same invention can also be used for the sampling of other body fluids, such as puss or venom.

Within a further embodiment the fluid sampling device, at least one, preferably two, of the vacuum sources is formed as a receptacle for accommodating body fluid. Therefore by activating the first vacuum source, the first vacuum source is fillable with the body fluid, thereby forming a first body fluid reservoir, and by activating the second vacuum source, the second vacuum source may be fillable with body fluid, thereby forming a second body fluid reservoir. Therefore, at least one vacuum source is adapted to be filled with the body fluid.

Alternatively, the fluid sampling device includes a tube interface, the tube interface adapted to interface at least one receptacle, the receptacle preferably being a sample receptacle or a medical analysis receptacle. Such a receptacle is therefore forming a body fluid reservoir. Therefore, the vacuum sources may be different from the at least one receptacle. This is advantageous due to the fact that the vacuum sources do not have to be formed as body fluid reservoirs. Such a medical analysis receptacle may be adapted to be filled with the sample of the body fluid for optionally performing a further analysis in a point of care or medical lab. A single vacuum source may be formed of one vacuum tube.

The first vacuum source may be interconnected to a first of the receptacles, and the second vacuum source may be interconnected to a second of the receptacles. Therefore, the activation of the first vacuum source activates the filling of the first receptacle. Afterwards by activation of the second vacuum source the second receptacle is fillable with body fluid.

According to the above at least one of the vacuum sources and/or at least one additional receptacle can form a body fluid reservoir.

In a further embodiment, the fluid sampling device includes an analysis arrangement, whereby the analysis arrangement is adapted to analyze the body fluid. Optionally the tube interface is connectable to the analysis arrangement.

A further embodiment of the fluid sampling device comprises a valve, preferably a stopcock, for changing the inlet from the at least first to an at least second body fluid reservoir, each of the body fluid reservoirs being formed of said vacuum source or said receptacle. Therefore, it is controllable how the body fluid reservoirs are fillable. To control which body fluid reservoir is fed, the valve can be turned into an appropriate position to allow the flow from the patient's wound to the chosen reservoir or receptacle respectively.

In yet a further embodiment of the fluid sampling device more than one body fluid reservoir may be used to store drawn body fluid. In an aspect of the embodiment the body fluid may be flowing into a first body fluid reservoir and subsequently into a second body fluid reservoir or even into further reservoirs. Switching from one reservoir to the next may be accomplished by means of a valve, preferably a stopcock.

A further embodiment of the fluid sampling device includes a watertight fluid extraction passage or conduit for connecting a patient's wound and the receptacle, the watertight fluid extraction passage or conduit respectively comprising a flexible tube. This is advantageous due to the fact that the disposable fluid sampling device is placeable on the body and operatable from a third person independently from the patient lying or standing.

A further embodiment of the fluid sampling device comprises a heating element which is attachable to the skin in the vicinity of an area, wherein the body fluid is drawn from the body. This simplifies the operability of the disposable fluid sampling device. Therefore, the skin of the patient around the position where the body fluid, especially blood, is drawn from the body of the patient, can be heated. That heating widens the blood capillaries in the immediate vicinity of the mentioned position, leading to a higher rate of blood drawable from the body of the person.

The heating element may be thin enough to be located between the blood collecting device and the patient's skin during the blood collection process, preferably between 0.5 mm and 4 mm, more preferably between 1 mm and 3 mm thick. Such a heating element may contain Capsicum extract, which is derived from chili peppers and helps to increase blood flow by lowering blood pressure and stimulating the release of nitric oxide and other substances that relax and widen the blood vessels, leading to a higher rate of blood drawable from the body of the person. Heat plasters containing capsicum are known in applications unrelated to blood collection (see, on the world wide web, for example ninelife.ch/products/hansaplast-on-heat-plaster-capsicumplaster-itensive-long-asting-4-sheets-x-12-total-48-sheets?gclid=EAIalQobChMIpazOsODPgAMVE-F3Ch3OEglmnEAQYASABEgIfzPD_BwE, the contents of which are incorporated herein by reference and relied upon, and shown in Appendix A).

Alternatively, rather than containing any active agents which stimulate the skin, a heating element may provide direct external heat for example by means of an exothermic chemical reaction. Such a reaction may be initiated for example by exposing the active agent(s) to air. The active agent(s) may for example contain iron powder or any other easily oxidizable substance which upon the exposure to air are oxidized and thereby produce heat. Other exothermic chemical reactions may be used, such as Calcium chloride or Magnesium sulfate dissolution in water, or Sodium Acetate crystallization.

Within a further embodiment of the fluid sampling device, the receptacle for accommodating the body fluid is made from translucent material, the translucent material preferably being colored and/or containing an additive adapted to contact the body fluid and after contact, changing the color without affecting measurable blood characteristics.

The body fluid reservoir may be made of translucent material and is preferably colored. Therefore, the color of the body fluid seen by the user may be different or even very different from the original color of the body fluid. The user may therefor see that the body fluid reservoir is filling, but because the color is no longer dark red, the non-dark-red color decreases the visual discomfort for people with blood phobia (a.k.a. hemophobia or hematophobia). Alternatively or additionally, the body fluid sampling device may contain an additive adapted to get in contact with the body fluid to be stored within the body fluid reservoirs so as to change the color of the body fluid without affecting its parameters or characteristics which are measured later on. Furthermore, instead of containing an additive which changes the color of the body fluid, the body fluid sampling device may comprise means present on the outside of the body fluid reservoirs which just seem to change the color of the body fluid without really changing it. Such a means may for example consist of a layer of cholesteric liquid crystal on the outside of the fluid reservoir, such liquid crystal changing color upon the filling of the fluid reservoir with warm body fluid.

The task is also solvable by a method for sampling body fluid, optionally self sampling, the method including the following steps:
a) in a first step, installing the fluid sampling device according to claim 1 on the patient's arm, the disposable fluid sampling device including a first vacuum source insertable into a vacuum interface;
b) in a second step, triggering the skin laceration or incision respectively for lacerating the skin;
c) in a third step, inserting a first vacuum source into the vacuum interface;
d) in a fourth step, filling a receptacle of the fluid sampling device with a body fluid; and
e) in a fifth step, removing the first vacuum source.

A further embodiment of the method includes a repetition of steps (c), (d), (e) executed for activating a second vacuum source of the fluid sampling device and optionally to fill a second receptacle with a sample of the body fluid.

Within a further embodiment of the method, a heating element of the body fluid sampling device is activated for a period of time, to keep the patient's skin at an elevated temperature in order to increase body fluid draw during the sampling process. Preferably the heating element remains activated during the entire sampling process.

The task can also be solved by a receptacle to be used with the disposable device, where a tamper evident label is connectable to a cap of the receptacle, so that be only opened in an non-tamper-evident manner at the lab performing the body fluid sample analysis, thereby allowing confirmation of the body fluid sample origin.

Within a further embodiment of the fluid sampling device, an extra vacuum source is used to collect the body fluid by providing vacuum to a non-vented first tube.

Within a further embodiment of the fluid sampling device, an amount of additives present in the receptacles for capillary sampling is adapted to the amount of body fluid to be collected. Typically 0.5 to 1.5 ml is the amount of body fluid to be collected.

Within a further embodiment of the fluid sampling device, the level of vacuum in the vacuum source is adapted to the need of capillary sampling, and so may differ from Annex B of a norm of ISO-6710.

Advantageously, laceration of the skin for body fluid sampling is made in a direction parallel to the receptacle, or parallel to the main length of the conduit or main fluidic passage leading the body fluid to the receptacle. This favors the process of blood collection.

Those skilled in the art will appreciate that elements in the Figures are illustrated for simplicity and clarity and have not necessarily been drawn to scale. For example, dimensions may be exaggerated relative to other elements to help improve understanding of the invention and its embodiments. Furthermore, when the terms ‘first’, ‘second’, and the like are used herein, their use is intended for distinguishing between similar elements and not necessarily for describing a sequential or chronological order. Moreover, relative terms like ‘front’, ‘back’, ‘top’ and ‘bottom’, and the like in the Description and/or in the claims are not necessarily used for describing exclusive relative position. Those skilled in the art will therefore understand that such terms may be interchangeable with other terms, and that the embodiments described herein are capable of operating in other orientations than those explicitly illustrated or otherwise described.

### BRIEF DESCRIPTION OF THE DRAWINGS

FIG. 1 is an image of the upper arm of a patient with an attached capillary blood sampling device.

FIG. 2 is an image of a capillary blood sampling device during the insertion of a vacuum tube.

FIG. 3 is an image of a capillary blood sampling device with inserted vacuum tube.

FIG. 4A is a top view of a capillary blood sampling device comprising a thin heating element.

FIG. 4B is a first side view of a capillary blood sampling device comprising a thin heating element.

FIG. 4C is a second side view (rear view of the first) of a capillary blood sampling device comprising a thin heating element.

FIG. 5 is a drawing of three sample tubes attached to a sampling device showing the subsequent filling of each.

FIG. 6 is a drawing of a capillary blood sampling device with a larger vacuum tube connected to a smaller sample tube.

FIG. 7 is a flowchart of a method of the invention, showing subsequent stages of removal and replacement of sample tubes.

FIG. 8A is an image of a sample tube comprising a cap with a tamper evident feature (TEF).

FIG. 8B is an image of various forms of sample tubes.

FIG. 9 is a drawing of a vent mechanism providing release of remaining vacuum after blood sampling.

FIG. 10 is an image of a patient's upper arm with incisions for blood sampling.

FIG. 11A is an image of a capillary blood sampling device with connexion to an external vacuum source.

FIG. 11B is an image of a capillary blood sampling device with connexion for a vacuum tube that is different from the blood sample tube.

FIG. 11C is schematic cross-section view of the inside of the sample tube connection when the vacuum source is different from the blood sample tube.

FIG. 12A to FIG. 12C shows an example of a mechanism of a lacerating device that can be used with the second and a third embodiments of the device of the invention.

FIG. 13A is an upper, perspective view of the first and second embodiments of the blood sampling device of the invention.

FIG. 13B is a lower, perspective view of the first and second embodiments of the blood sampling device of the invention.

FIG. 14A is an upper, perspective view of the third embodiment of the body fluid sampling device of the invention before use.

FIG. 14B is a lower, perspective view of the third embodiment of the body fluid sampling device of the invention before use.

FIG. 14C is a top view of the third embodiment of the body fluid sampling device of the invention before use.

FIG. 14D is a bottom view of the third embodiment of the body fluid sampling device of the invention before use.

FIG. 14E is a left side view of the third embodiment of the body fluid sampling device of the invention before use.

FIG. 14F is a right side view of the third embodiment of the body fluid sampling device of the invention before use.

FIG. 14G is a front side view of the third embodiment of the body fluid sampling device of the invention before use.

FIG. 14H is a back side view of the third embodiment of the body fluid sampling device of the invention before use.

FIG. 14I is an upper, perspective view of the third embodiment of the body fluid sampling device of the invention while in use.

FIG. 14J is a lower, perspective view of the third embodiment of the body fluid sampling device of the invention while in use.

FIG. 14K is a top view of the third embodiment of the body fluid sampling device of the invention while in use.

FIG. 14L is a bottom view of the third embodiment of the body fluid sampling device of the invention while in use.

FIG. 14M is a left side view of the third embodiment of the body fluid sampling device of the invention while in use.

FIG. 14N is a right side view of the third embodiment of the body fluid sampling device of the invention while in use.

FIG. 140 is a front side view of the third embodiment of the body fluid sampling device of the invention while in use.

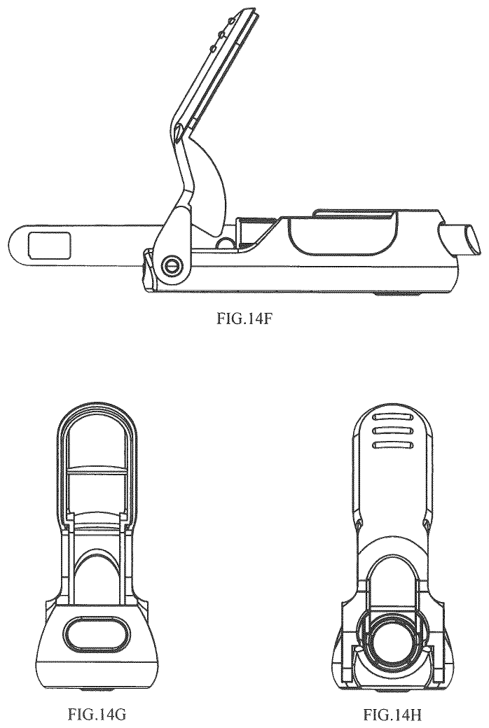

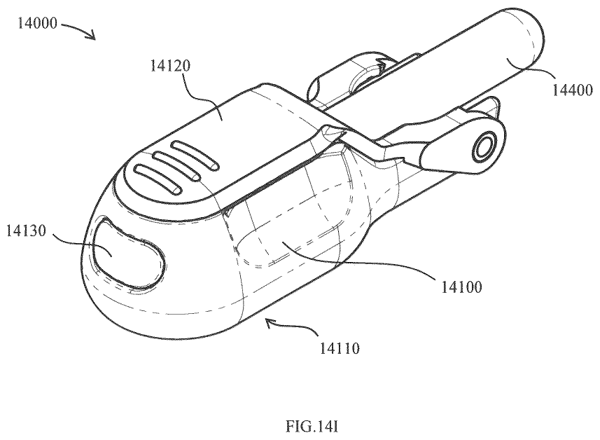

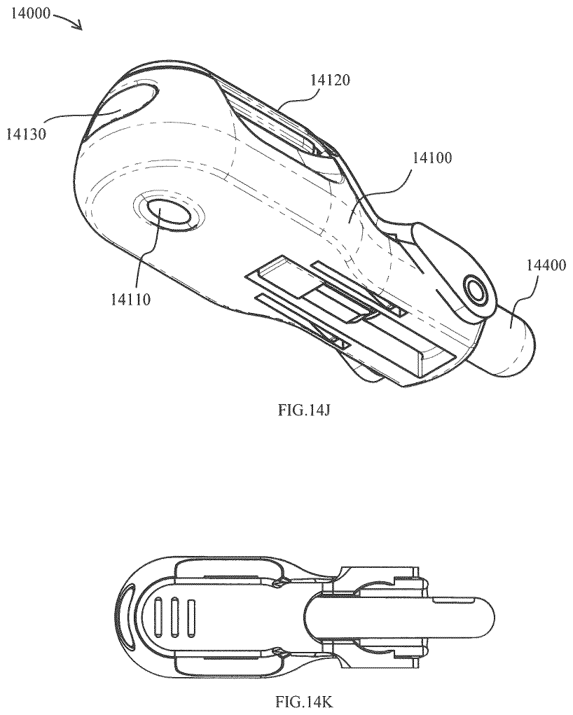

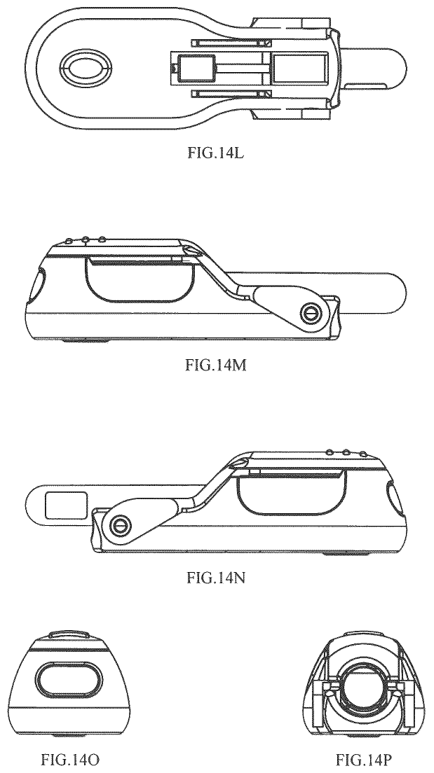

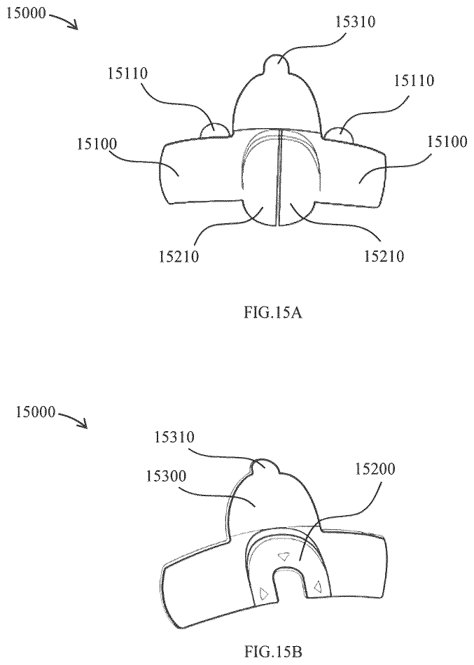

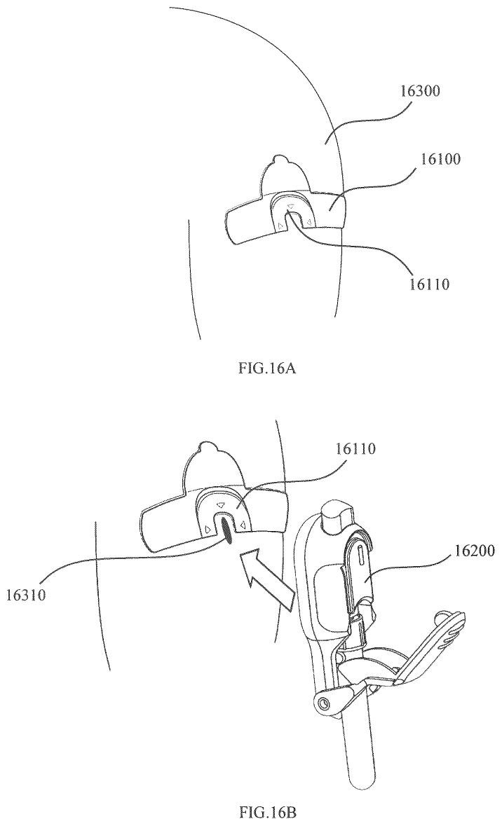

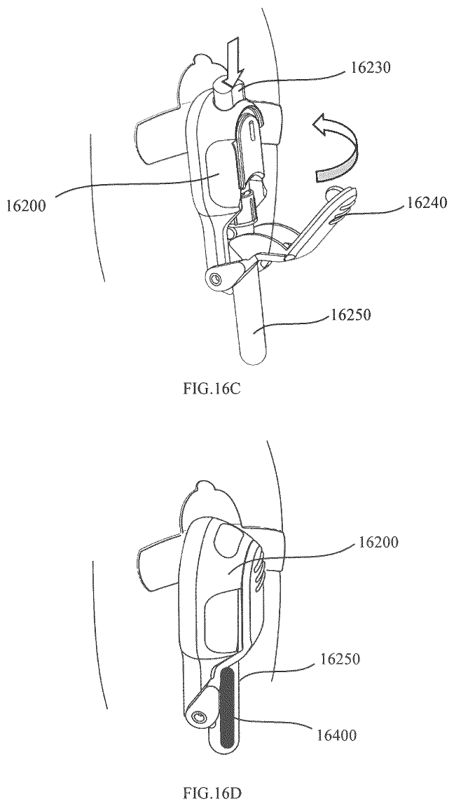

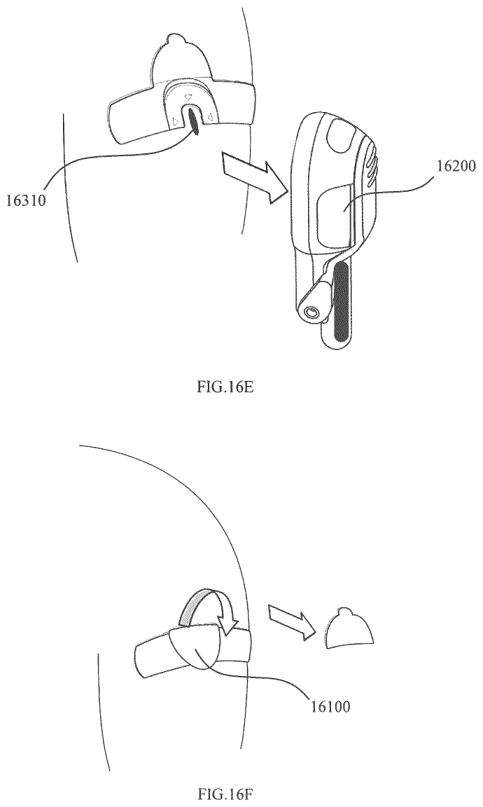

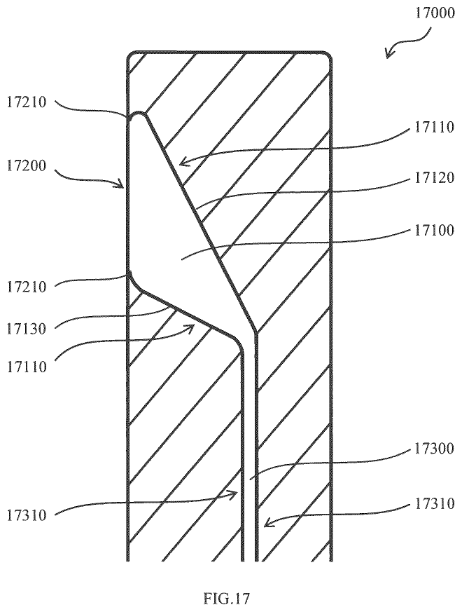

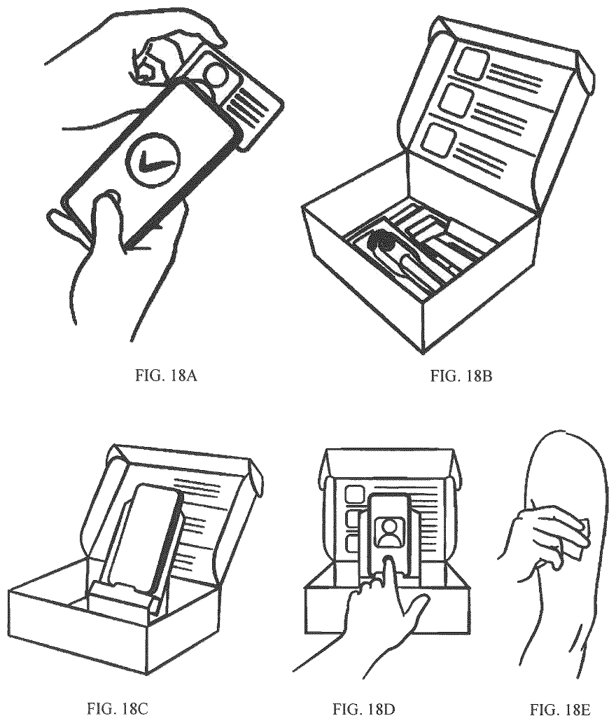

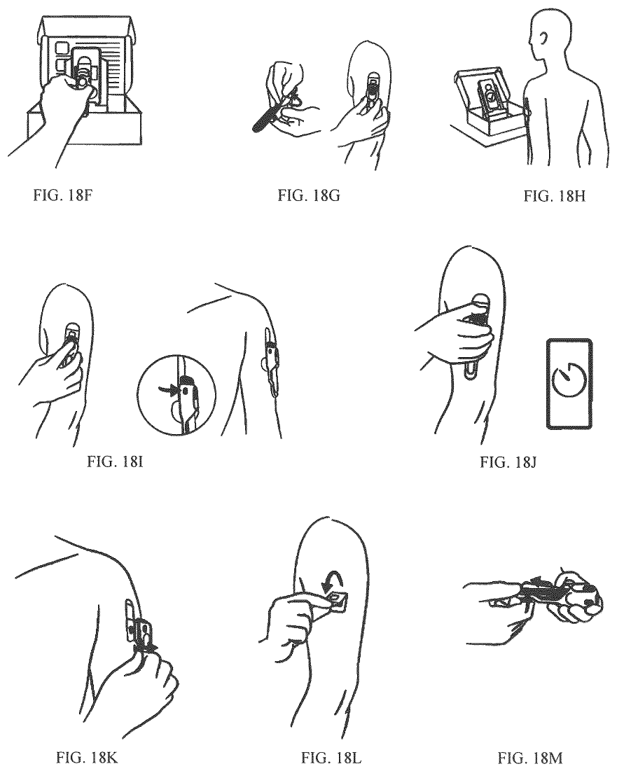

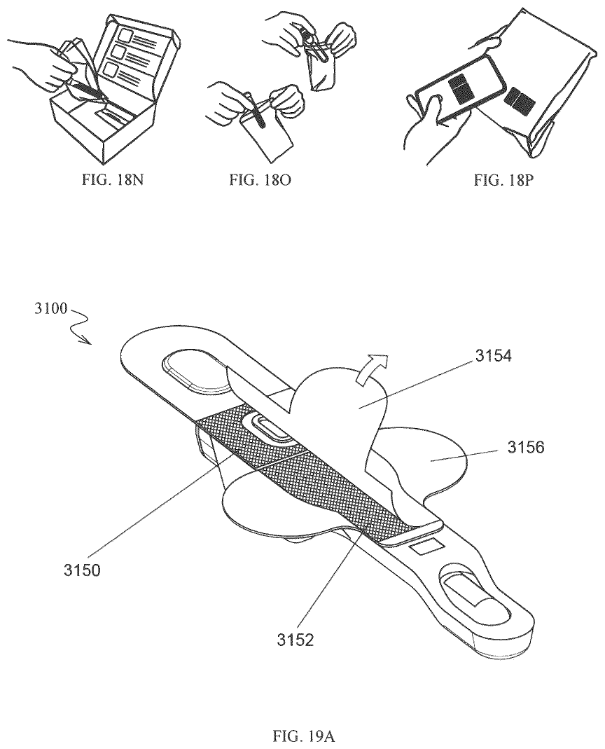

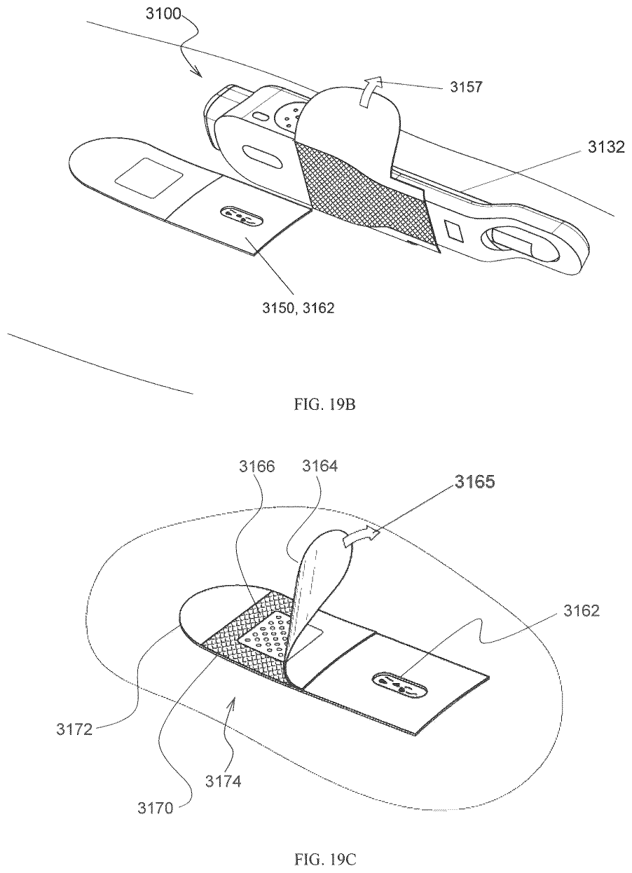

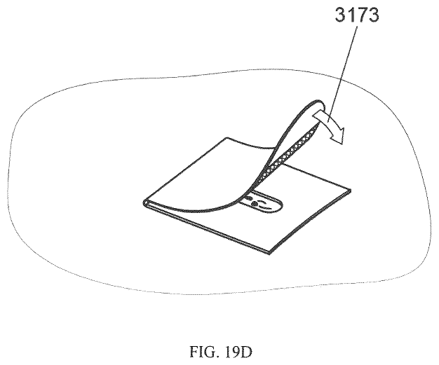

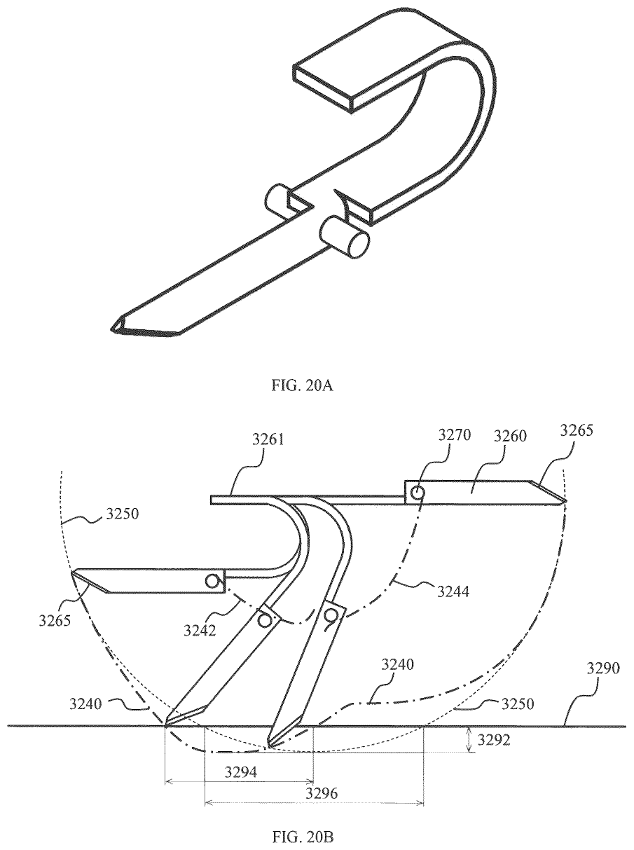

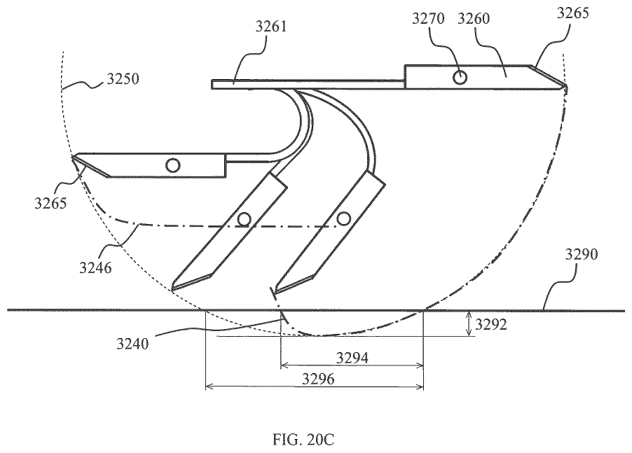

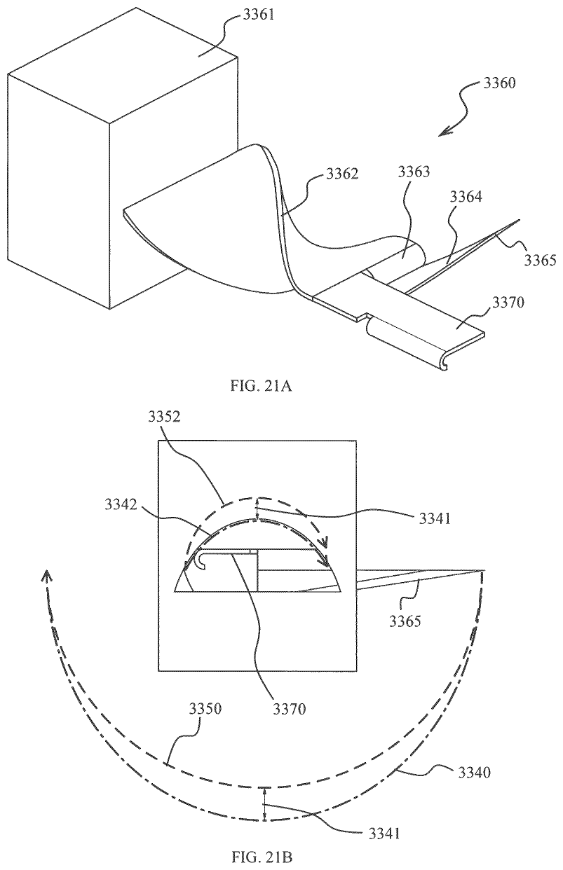

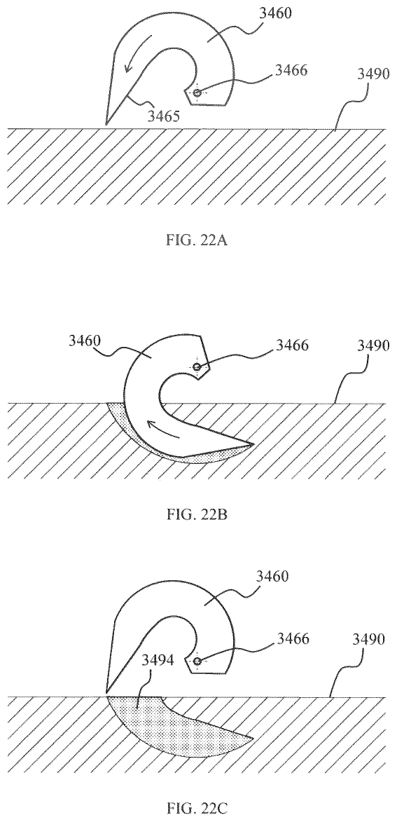

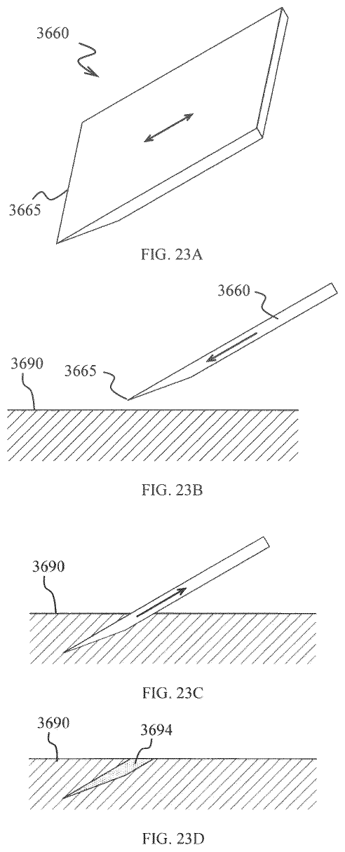

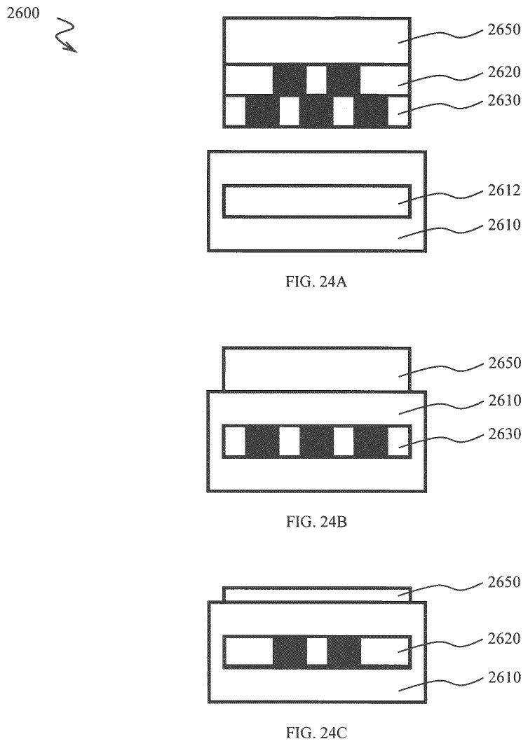

FIG. 14P is a back side view of the third embodiment of the body fluid sampling device of the invention while in use.

FIG. 15A shows an adhesive pad of the invention before use.

FIG. 15B shows an adhesive pad of the invention while in use.

FIG. 16A illustrates the step of installing the adhesive pad of the invention.

FIG. 16B illustrates the step of installing the fluid sampling device of the invention.

FIG. 16C illustrates the step of activating the sampling with the device of the invention.

FIG. 16D illustrates the step of waiting and monitoring the sampling with the device of the invention.

FIG. 16E illustrates the step of removing the device of the invention after sampling.

FIG. 16F illustrates the step of dressing the wound with the adhesive pad of the invention.

FIG. 17 shows a cross section lateral view of the suction chamber of the fluid sampling device of the invention.

FIGS. 18A to 18P are schematic views illustrating the steps of a fourth embodiment of the method of using the system of the invention.

FIGS. 19A to 19D are schematic views with more detail about the specific features of the adhesive integrated dressing included in the capillary blood sampling device.

FIG. 20A is a perspective view of a fourth embodiment of the cutting blade of a device of the invention is made of a single part.

FIG. 20B is a side, four-position, shutter view of the fourth embodiment of the cutting blade of a device of the invention.

FIG. 20C is a side, four-position, shutter view of the fourth embodiment of the cutting blade of a device of the invention showing the retraction thereof.

FIG. 21A is a perspective view of a fifth embodiment of the cutting blade of the invention.

FIG. 21B is a front view of the fifth embodiment of the cutting blade of the invention.

FIG. 22A is a side view of a sixth embodiment the cutting blade of a device of the invention, prior to entry into the epidermal layer of the patient's body.

FIG. 22B is a side view of the sixth embodiment the cutting blade of a device of the invention half way through its cycle of motion.

FIG. 22C is a side view of the sixth embodiment the cutting blade of a device of the invention at the end of its cycle of motion, having returned to its initial position.

FIG. 23A is a perspective view of a seventh embodiment the cutting blade of a device of the invention showing a standard scalpel blade.

FIG. 23B is a side view of the seventh embodiment the cutting blade of a device of the invention, prior to entry into the epidermal layer.

FIG. 23C is a side view of the seventh embodiment of the cutting blade of a device of the invention after entry into the epidermal layer, as it is about to be removed from said layer.

FIG. 23D is a side view of the epidermal layer showing the cut made by the seventh embodiment of the cutting blade.

FIG. 24A to FIG. 24C show examples of visible features that can be integrated in a device of the invention to be easily recognized by the human observer, or easily identified by a real-time image analysis software.

### DETAILED DESCRIPTION OF THE PREFERRED EMBODIMENT

The following description is not intended to limit the scope of the invention in any way as they are exemplary in nature, serving to describe the best mode of the invention known the inventors as of the filing date hereof. Consequently, changes may be made in the arrangement and/or function of any of the elements described in the exemplary embodiments disclosed herein without departing from the spirit and scope of the invention.

Note that in this application, where ever blood or capillary blood sampling is mentioned, it should be understood that puss or venom can be substituted for blood.

Note also that the terms body fluid reservoir, fluid reservoir, vacuum tube, sample tube and analysis tube may refer to the same object, depending on the details of the body fluid sampling device of the invention.

As the sampling device may be used by non-trained users, even in some cases for auto-sampling, the main parts to be manipulated by the user may be color-coded for easier instructions dispensing and recognition, as mentioned below.

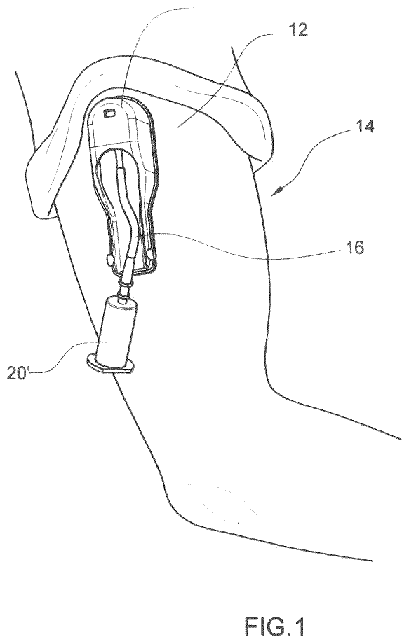

Referring now to FIGS. 1 to 3, a first embodiment of a capillary body fluid sampling device comprises (a) a capillary blood sampling device 10 according, for example, to PCT/IB2021/000187 and/or PCT/IB2021/000580, the contents of which are incorporated herein by reference and relied upon, the capillary blood sampling device 10 being shown mounted against the skin 12 of a patient 14. A flexible tube 16 is disposed between the blood sampling device 10 and a vacuum tube 20, which in operation is inserted in a vacuum tube connector 20′ or an external vacuum source, thereby providing a watertight extraction passage or conduit between the patient's wound 100 and the sample tube (vacuum tube 20). This arrangement allows for easy insertion/removal of one or more vacuum tubes in a row without risking destabilizing or detaching the blood sampler from the patient's skin 12.

The level of vacuum in the vacuum tube or in the external vacuum source is adapted to the need of capillary sampling, and may differ from the Annex B of ISO-6710 norm.

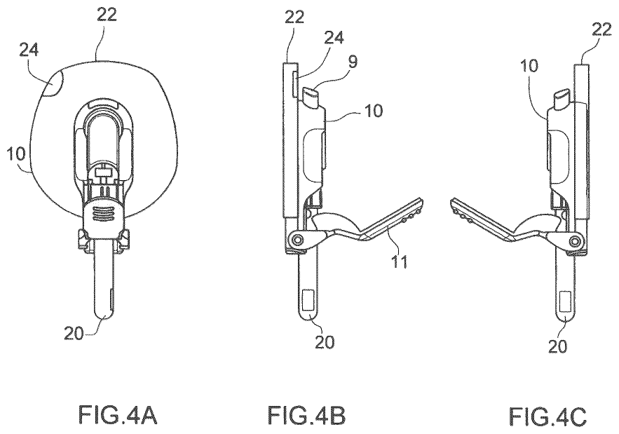

Referring now to FIG. 4A, a second embodiment of the fluid sampling device 10, which is optionally disposable, optionally includes a heating element 22. Preferably, the heating element 22 has a thickness in a range of about 0.5 to 4 mm, or even more preferably about 1 to 3 mm. The fluid sampling device 10 with the heating element 22 is shown here in a top view. The heating element 22 is disposed to provide heating of the area around the patient's wound from which the body fluid, preferably blood, is to be drawn. The fluid sampling device is attachable to the skin 12 and so when the skin 12 is heated with the help of the heating element 22, the capillaries are widened, so that more body fluid can be drawn from the body. Note that here, the vacuum source 20 or external vacuum source is optionally directly connected to the fluid sampling device 10 and not via a flexible tube 16.

Referring now to FIGS. 4B and 4C the fluid sampling device 10 of the second embodiment, which is optionally disposable and comprises the optional heating element 22, is shown in two side views. The heating element 22 is locatable in direct contact to the skin of the patient, i.e. on the side of the patient's skin In other words, the heating element 22 is thin enough to be located between the blood collecting device and the patient's skin during the blood collection process. Such a heating element 22 may contain Capsicum extract, which is derived from chili peppers and helps to increase blood flow by lowering blood pressure and stimulating the release of nitric oxide and other substances that relax and widen the blood vessels, leading to a higher rate of blood drawable from the body of the person. When attached to the skin 12 and heated, the capillaries are widened, so more blood can be drawn from the body.

In an aspect of the embodiment, rather than containing any reactive agents which stimulate the skin, a heating element 22 may provide direct external heat for example by means of an exothermic chemical reaction. Such a reaction may be initiated for example by exposing the reactive agent(s) to air. The reactive agent(s) may for example contain iron powder or any other easily oxidizable substance which upon the exposure to air are oxidized and thereby produce heat. The oxidation of the oxidizable substance and hence the initiation of the heating may optionally be initiated by lifting a flap 24 on heating element 22, thereby letting air penetrate into the oxidizable substance.

In another aspect of the embodiment a heating element 22 may consist of at least two compartments the oxidizable substances in which can separately be exposed to air by successively drawing flaps 24 in order to keep a patient's skin at an optimal temperature for an extended time in which blood can be drawn during a sampling process. Other exothermic chemical reactions may be used, such as Calcium chloride or Magnesium sulfate dissolution in water, or Sodium Acetate crystallization.

As mentioned above, he main parts to be manipulated by the user may be color-coded for easier instructions dispensing and recognition. For example the lever 11 may be colored in yellow and the push-button 9 may be colored in green, while the sampling device itself may be essentially white. It should be understood however that other color codes providing sufficient contrast for the same purpose may be used.

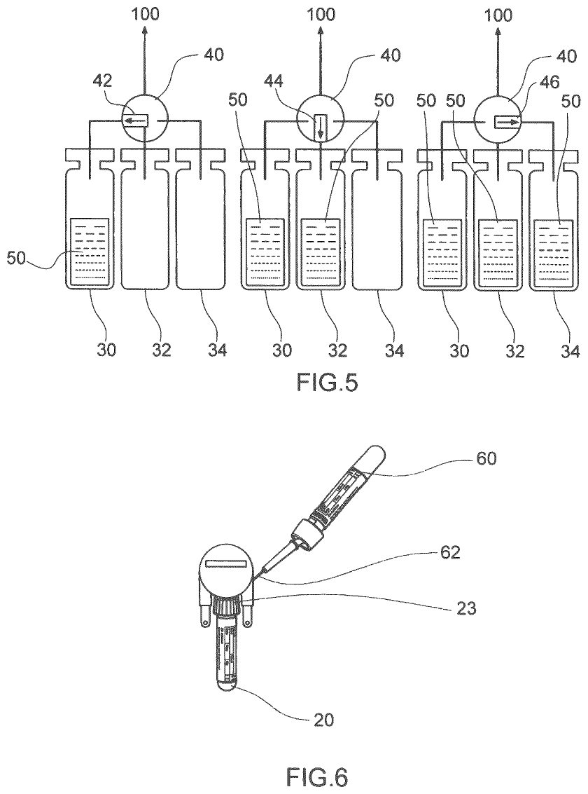

Referring now to FIG. 5, in a third embodiment of the fluid sampling device 10 for example three body fluid reservoirs 30, 32, 34 are connected to a valve 40 communicating with the wound 100 in the patient's skin 12 from which the body fluid is to be drawn. The valve 40 may consist of a stopcock. The body fluid 50 drawn from the wound 100 in the patient's skin 12 can flow into one of the body fluid reservoirs 30, 32, 34 at a time. To control which body fluid reservoir 30, 32, 34 is fed, the valve 40 can be turned. On the left of FIG. 5, the valve is in a position 42, such that only the first fluid reservoir 30 is filled. In the middle of FIG. 5 the valve 40 has been turned to a position 44, such that the second fluid reservoirs 32 is filled. Finally, on the right of FIG. 5 all three reservoirs 30, 32, 34 are filled, after the valve 40 has been turned into position 46. The body fluid reservoirs 30, 32, 34 described in all embodiments here need not be identical and so they may be of different sizes and/or colors. The identifiable first receptacle may contain specific reactants different from the reactants in the other receptacles, permitting certain tests to be carried out on the contents of the first receptacle.

Referring to FIG. 6, a fourth embodiment of a fluid sampling device 10 comprises two optionally different receptacles. A first receptacle 20 may be smaller receptacle already validated by the FDA for capillary blood sampling, and so is expected to be easier to get through FDA approval. A larger second receptacle 60, connected via a flexible tube 62 adapted to pass through the septum 23 of the first receptacle 20, creates vacuum in the device and in the smaller first receptacle and thereby helps filling the smaller first receptacle 20.

In all four embodiments of the fluid sampling device 10, the incision or laceration of the skin made for blood sampling is preferably made in a direction parallel to a sample receptacle 20, or parallel to the main length of the conduit 16 or main fluidic passage leading the body fluid to the receptacle. This favors multiple incisions to be made for faster capillary blood sampling.

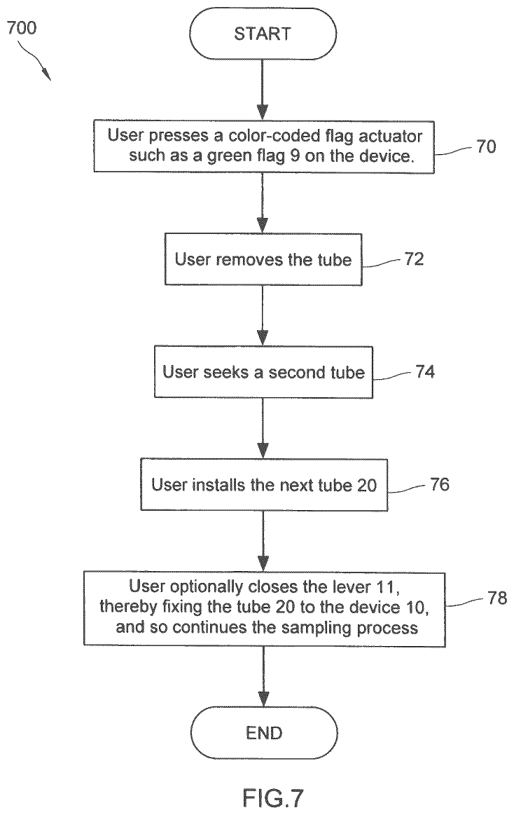

Referring now to FIG. 7, steps of a method 700 for sample receptacle 20 removal and/or replacement are shown. In a first step 70, the user presses a color-coded flag actuator such as a green flag 9 of the fluid sampling device 10 to lift up the lever 11 fixing receptacle 20. Pushing the green button 9 disengages the tube 20 from the hollow needle (so that the septum closes) and lifts the lever 11, so that the tube 20 can be removed and replaced by the next tube. The “color-coded flag” aspect of the actuator helps make it easier to spot by the user/patient in case of self-sampling, such as by authentification software such as mentioned in PCT application PCT/US2021/25087 filed Mar. 31, 2023, the content of which is incorporated herein in its entirety and relied upon. In a second step 72, the user removes the receptacle 20. In a third step 74, the user seeks a second receptacle 20. In a fourth step 76, the user installs the next receptacle 20. In a fifth step 78, the user optionally closes the lever 11, thereby fixing the receptacle 20 and pushing out the color-coded flag actuator 9, and so continues the sampling process.

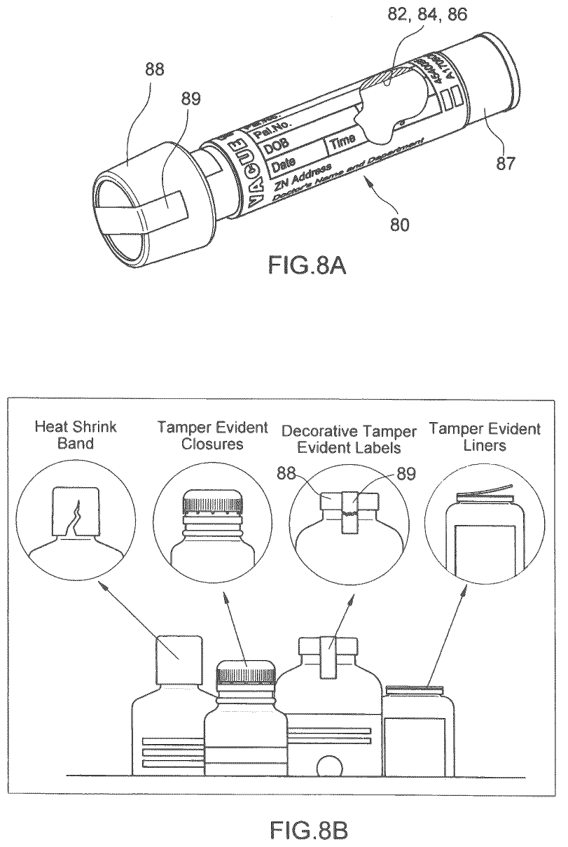

Referring now to FIG. 8A, an embodiment of a sample receptacle 80 optionally has a size about twice as large as the volume of body fluid, preferably blood, to be collected (e.g. 4 ml tube volume to collect 2 ml of blood). Optionally, the sample receptacle 80 of the embodiment provides a excess vacuum beyond the vacuum level needed to fill the sample receptacle 80 in order to speed up the blood collection process (for example from −0.85 bar to −0.30 bar). The size of the sample receptacle 80 and the amount of excess vacuum are particularly adapted for drawing blood from the capillaries in a patient's skin, as blood drawn from skin capillaries does not flow as fast as blood drawn from a punctured vein. The vacuum level in the sample receptacle 80 may differ from the standard phlebotomy norm, given its larger volume, depending on the volume of blood to be collected and/or on the sensitivity of a patient to suction pressure.

Optionally, the interior of the sample receptacle 80 has a coating 82 to avoid blood coagulation (for example a heparin coating or or a similar coating). The sample receptacle 80 may also contain additives 84 and/or a separation gel 86 for the conservation/transport of blood cells separate from the plasma.

Referring now again to FIG. 8A the sample receptacle 80 is typically closed by a cap or stopper 88. Optionally the cap 88 may be provided with a tamper evident feature (TEF) 89 (additional feature to ensure blood is the one of the user) may advantageously be provided. With such a feature 89, only a laboratory may open the sample receptacle. The laboratory can check if the tube was tampered with prior to testing its contents. The TEF 89 also marks the tube 80 with a mark 87 during blood collection process, which shows that this tube has already been in use.

Referring now to FIG. 8B, various anti-tampering features are useful for blood collection or body fluid collection at home. Such anti-tampering features include for example a plastic covering of the tube cap or a tape on the tube to cap junction.

In an aspect of the embodiment the body fluid reservoir 20, 80 may include a body 87 made of color changing and/or translucent and/or opaque, and/or thermocromic material such as glass, colored glass, PET, colored PET, PET charged with thermochromic dyes. Therefore, the color of the body fluid seen by the user may be different or even very different from the original color of the body fluid. The user may therefor see that the body fluid reservoir is filling, but because the color is no longer dark red, the non-dark-red color decreases the visual discomfort for people with blood phobia (a.k.a. hemophobia or hematophobia). Alternatively or additionally, the body fluid sampling device may contain an additive adapted to get in contact with the body fluid to be stored within the body fluid reservoirs so as to change the color of the body fluid without affecting its parameters or characteristics which are measured later on. Furthermore, instead of containing an additive which changes the color of the body fluid, the body fluid sampling device may comprise means present on the outside of the body fluid reservoirs which just seem to change the color of the body fluid without really changing it. Such a means may for example consist of a layer of cholesteric liquid crystal on the outside of the fluid reservoir, such liquid crystal changing color upon the filling of the fluid reservoir with warm body fluid.

The amount of any eventual additives present in the body fluid reservoir for capillary sampling is adapted to the amount of body fluid 50 to be collected (0.5-1.5 ml).

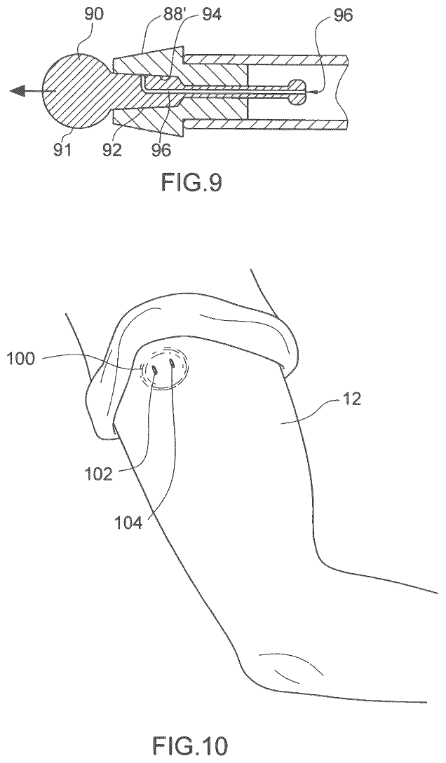

Referring now to FIG. 9, in an aspect of the embodiment of a sample receptacle 80, a vent 90 may optionally provided to release any remaining vacuum after blood sampling, thereby reducing the risk of hemolysis during conservation or transport. The vent 90 can be pulled axially away from the cap 88′, such as to separate a vent surface 92 from a corresponding cap surface 94, thereby releasing any remaining vacuum through a passageway 96 in the vent 90. A knob 91 can be grasped by the user with her/his fingers in order to pull the vent 90, separating surfaces 92, 94 and thereby release the remaining vacuum.

Referring now to FIG. 10, a wound 100 for blood collection example in a patient's upper arm 12 may consist of at least one incision or laceration 102, 104 formed parallel to the direction of blood collection/sample receptacle, or parallel to the main length of the conduit 16 or main fluidic passage leading the body fluid to the receptacle. The laceration in the direction of blood collection is particularly advantageous when more than one incision 102, 104 is to be made for faster capillary blood flow, because in case of laceration perpendicular to the direction of the blood flow, blood flowing from the upper incision would flow over the lower incision.

### Method of Use

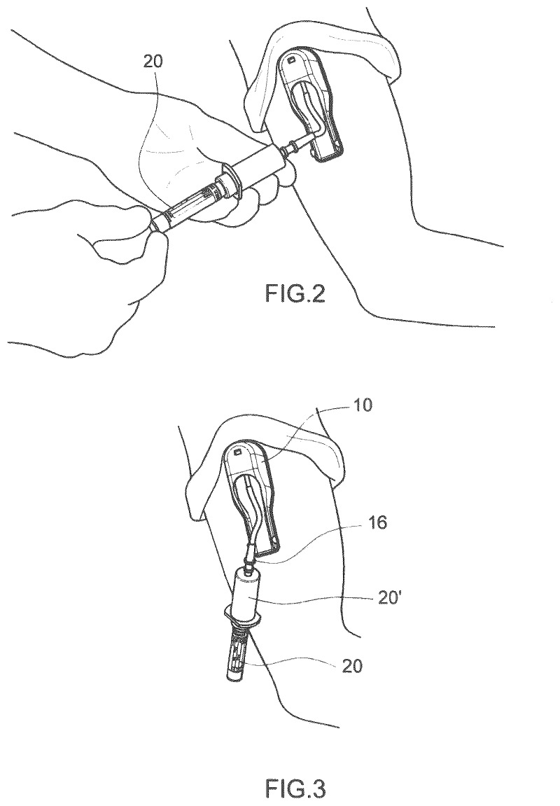

1. FIG. 1: The user (medical personnel or untrained person) installs the fluid sampling device 10 on the patient's skin 12 with an adhesive pad (such as a a thin heating element 22 but one which does not need to be heated). Any other appropriate means known in the industry may be used to install the fluid sampling device 10. It may for example simply be held or pressed against the skin by the patient himself or by a third person)
2. The user triggers the skin incision or laceration
3. FIG. 2: The user inserts a vacuum source 20 into the connector 20′ (irrespective of being connected to the device via a flexible tube 16) (N.B: steps 2 and 3 may also be executed in the reverse order)
4. FIG. 3: The vacuum tube 20 is filling with the body fluid 50, typically with blood.
5. The user removes the vacuum source 20
6. Optionally the user inserts a second vacuum source 20, repeating steps 4 to 6 as many times as necessary
7. The user removes the device 10 from the patient's skin 12, and cleans and dresses the wound
8. The user sends the sample tube(s) 20 to the laboratory for analyses

In any of the embodiments mentioned herein, where the sample tube 20 is different from the vacuum tube 60 (FIG. 6), the user also changes the sample tube when he changes the vacuum tube.

In any of the embodiments mentioned herein, where there is more than one sample tube 30, 32, 34 connected to the sampling device 10 and the vacuum source is more than one vacuum tube (a combination of FIG. 5 and FIG. 6), the user may change the vacuum tube 60 to have a fresh vacuum tube to fill each sample tubes (30, 32, 34), even without the need of a valve when the tubes are swapped in a certain order. If a valve were to be used, it would be advantageously placed at the T-junction between tubes 112 and 116.

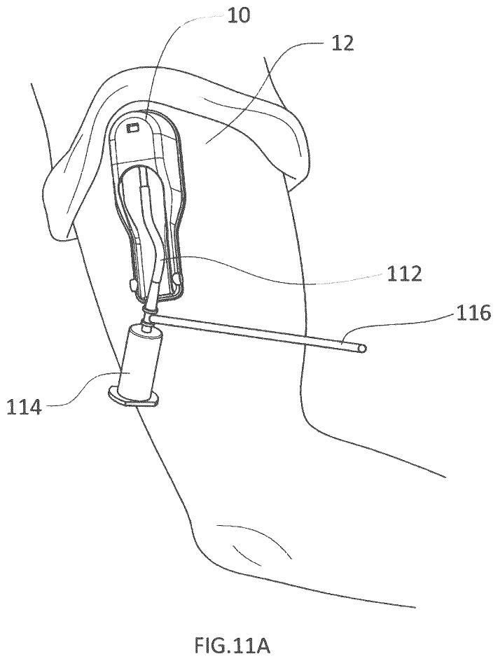

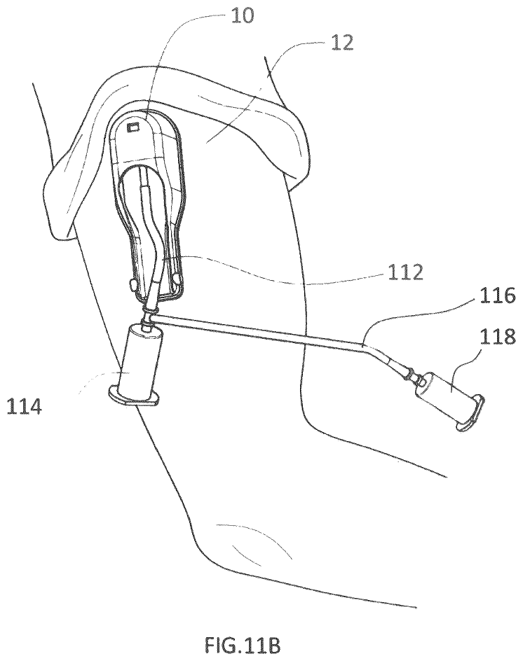

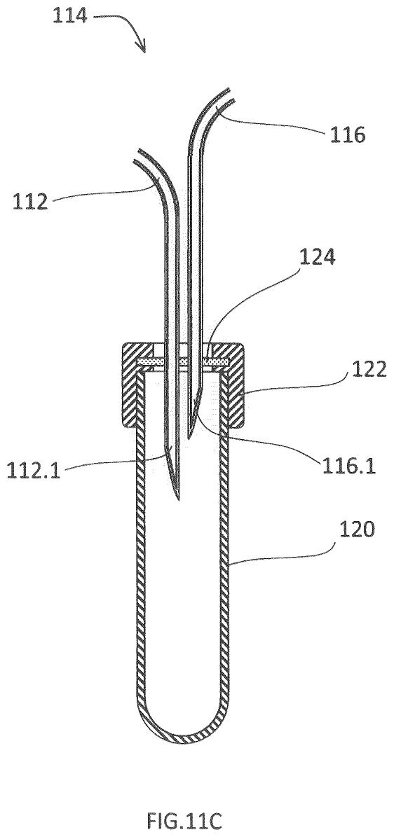

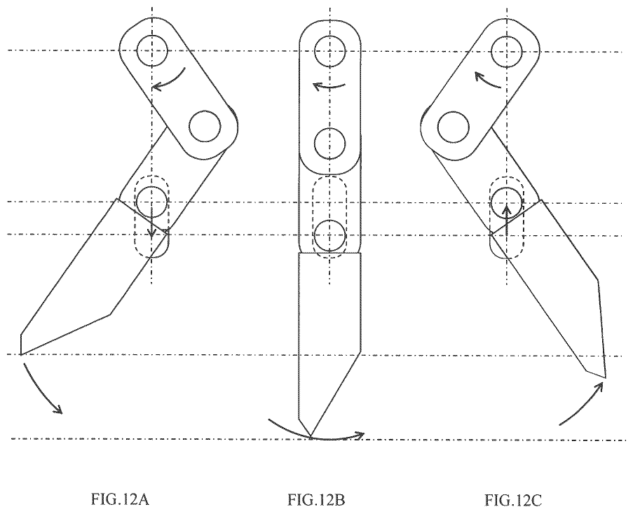

Referring now to FIGS. 11A, the fluid sampling device 10 includes a connector 114 connected to the device 10 via a flexible tube 112 to receive the blood collection/sample receptacle, and a conduit 116 to connect the receptacle to a vacuum source, as usually available in medical centers. In such configurations, the user can connect sequentially several receptacles to the connector 114, thereby providing more than one receptacle containing a blood sample.

Referring now to FIGS. 11B, the fluid sampling device 10 includes a connector 114 connected to the device 10 via a flexible tube 112 to receive the blood collection/sample receptacle, and a second connector 118 connected to the receptacle via a conduit 116 to connect to a vacuum tube 20. In such a configurations, after the fluid sampling device 10 has been being installed on the patient's skin 12, the user executes the following steps:
1. In a first step, connecting a first receptacle to the connector 114
2. In a second step, connecting a first vacuum tube to the second connector 118.
3. In a third step, triggering the skin incision or laceration (step 3 may also be executed before the first step or before the second step)
4. In a fourth step, waiting for the first receptacle to be filled with blood
5. In a fifth step, disconnecting the first vacuum tube from the second connector 118
6. In a sixth step, disconnecting the first receptacle from the connector 114
7. In a seventh step, connecting a second receptacle to the connector 114
8. In an eighth step, connecting a second vacuum tube to the second connector 118
9. In a ninth step, repeating steps 4 to 8, respectively steps 4 to 6 until having obtained a sufficient number of receptacles filled with a blood sample.

Of course a configuration in which a valve is used to connect several receptacles, respectively several vacuum tubes, in a row similar to the configuration described in FIG. 5 may also be used.

Referring now to FIG. 11C, for both configurations shown in FIG. 11A and FIG. 11B, the junction between tubes 112 and 116 may resemble externally a T-junction, but the internal configuration details are provided here. Functionally, the junction between tubes 112 and 116 is advantageaously made in the connector 114 through the blood sample tube. The connector 114 provides a first connection for the device 10 through the flexible tube 112 into the sample tube 120 via a hollow needle 112.1 able to pierce the septum 124, held in the cap 122 that closes the sample tube 120. The connector 114 also provides a second connection for the vacuum source through the conduit 116 via a hollow needle 116.1 able to pierce the septum 124, held in the cap 122 that closes the sample tube 120. Therefore, when a fresh vacuum tube is inserted into the connector 118, or when the vacuum source is activated directly or via the opening of a valve, the vacuum is established first within the sample tube 120, and then progresses through the flexible tube 112 until the sampling device 10 to provide the necessary suction for collecting the blood into the sample tube 120.

Specific embodiments of the invention can be summarized belonging to at least one of the following feature sets:

1. A disposable fluid sampling device including:
(a) body fluid sampling means, optionally body fluid sampling means for auto-sampling;
(b) optionally, analysis means using one or more droplet(s) of the sampled fluid to analyze the fluid; and
(c) a means for at least one medical body fluid reservoir, body fluid sample tube or analysis tube, the at least one tube adapted to be filled with a sample of fluid for analysis in a point of care or medical lab,
wherein the means includes an interface for at least one vacuum tube or external vacuum source, the vacuum tube or external vacuum source being adapted for providing suction necessary to fill the at least one body fluid sample tube with the fluid.
   2. The disposable fluid sampling device of feature set 1, wherein a watertight fluid extraction passage or conduit from the patient's wound to the body fluid sample tube comprises a flexible tube.
   3. The disposable fluid sampling device of feature set 1, wherein a thin heating element is disposed to provide heating around the area around the patient's wound from which blood is to be drawn. The thin heating element may have a thickness in a range of about 0.5 to 4 mm, or preferably about 1 to 3 mm. When the sampling device is attached to the skin and heated, the capillaries are therefore widened, so that more blood can be drawn from the body. The vacuum tube or external vacuum source may either directly be connected to the sampling device or via a flexible tube, both providing a watertight fluid extraction passage or conduit from the patient's wound to the body fluid sample tube.
   4, The thin heating element of feature set 3, wherein said heating element contains at least one of the following ingredients:
(a) A Capsicum extract, which is derived from chili peppers and helps to increase blood flow by lowering blood pressure and stimulating the release of nitric oxide and other substances that relax and widen the blood vessels, leading to a higher rate of blood drawable from the body of the person,
(b) An reactive agent providing an exothermic chemical reaction. The reactive agent may for example contain iron powder or any other easily oxidizable substances which upon the exposure to air are oxidized and thereby produce heat. Other exothermic chemical reactions may be used, such as Calcium chloride or Magnesium sulfate dissolution in water, or Sodium Acetate Crystallization. The exothermic reaction may for example be initiated by pulling a flap by means of which the reactive agent is no longer isolated from the ambient air.
   5. The disposable fluid sampling device of feature set 1, wherein more than one, for example three tubes are connected to a valve communicating with the patient's skin from which blood is to be drawn. The valve may consist of a stopcock. The body fluid drawn from the patient's skin can flow into at least one of the body fluid reservoirs at a time. To control which body fluid reservoir is fed, the valve can be turned. The body fluid reservoirs need not be identical, they may have different sizes and/or colors. The identifiable first tube may contain specific reactants different from the reactants in the other tubes, permitting certain tests to be carried out on the contents of the first tube.
   6. The disposable fluid sampling device of feature set 1, wherein for example two optionally different tubes are used. A first tube may be a smaller tube already validated by the FDA for capillary blood sampling, and so is expected to be easier to get through FDA approval. A larger tube, connected via a flexible tube adapted to pass through the septum of the first tube, creates vacuum in the device and in the small tube and thereby helps filling the small tube.
   7. The disposable fluid sampling device of feature set 1, wherein a sample tube optionally has a size about twice as large as the volume of blood to be collected (e.g. 4 ml tube volume to collect 2 ml of blood). Optionally, this sample tube provides a excess vacuum beyond the vacuum level needed for it to be filled in order to speed up the blood collection process (for example from −0.85 bar to −0.30 bar). The size of the sample tube and the amount of excess vacuum are particularly adapted for drawing blood from the capillaries in a patient's skin, as blood drawn from skin capillaries does not flow as fast as blood drawn from a punctured vein.
   The vacuum level in the sample tube may differ from the standard phlebotomy norm, given its larger volume, depending on the volume of blood to be collected and/or on the sensitivity of a patient to suction pressure.

Optionally, the interior of the sample tube has a coating to avoid blood coagulation (for example a heparin coating or or a similar coating). The sample tube may also contain additives and/or a separation gel for the conservation/transport of blood cells separate from the plasma.

The sample tube is typically closed by a cap or stopper. Optionally the cap may be provided with a tamper evident feature (TEF) (additional feature to ensure blood is the one of the user) may advantageously be provided. With such a feature, only a laboratory may open the sample tube. The laboratory can check if the tube was tampered with prior to testing its contents. The TEF also marks the tube with a mark during blood collection process, which shows that this tube has already been in use. Various anti-tampering features are useful for blood collection or body fluid collection at home. Such anti-tampering features include for example a plastic covering of the tube cap or a tape on the tube to cap junction.

8. The disposable fluid sampling device of feature set 1, wherein the body fluid reservoir includes a body made of color changing and/or translucent and/or opaque, and/or thermocromic material such as glass, colored glass, PET, colored PET, PET charged with thermocromic dyes. Therefore, the color of the body fluid seen by the user may be different or even very different from the original color of the body fluid. The user may therefor see that the body fluid reservoir is filling, but because the color is no longer dark red, the non-dark-red color decreases the visual discomfort for people with blood phobia (a.k.a. hemophobia or hematophobia). Alternatively or additionally, the body fluid sampling device may contain an additive adapted to get in contact with the body fluid to be stored within the body fluid reservoirs so as to change the color of the body fluid without affecting its parameters or characteristics which are measured later on. Furthermore, instead of containing an additive which changes the color of the body fluid, the body fluid sampling device may comprise means present on the outside of the body fluid reservoirs which just seem to change the color of the body fluid without really changing it. Such a means may for example consist of a layer of cholesteric liquid crystal on the outside of the fluid reservoir, such liquid crystal changing color upon the filling of the fluid reservoir with warm body fluid.

The amount of any eventual additives present in the body fluid reservoir for capillary sampling is adapted to the amount of body fluid to be collected (0.5-1.5 ml).

9. The disposable fluid sampling device of feature set 1, wherein a vent provides the release of any remaining vacuum after blood sampling, thereby reducing the risk of hemolysis during conservation or transport. The vent can be pulled axially away from the cap of sample tube, such as to separate a vent surface from a corresponding cap surface, thereby releasing any remaining vacuum through a passageway in the vent. A knob can be grasped by the user with her/his fingers in order to pull the vent, separating vent and cap surfaces and thereby releasing the remaining vacuum.
10. A method of capillary blood sampling, optionally self sampling, the method including at least the steps of:
a) In a first step, preparing the area of the patient's arm where the blood sample will be taken, according to the standards of the profession (preheating, shaving, disinfection, etc.)
b) In a second step, installing the device on the patient's arm
c) In a third step, triggering the skin laceration
d) In a fourth step, inserting a vacuum tube in the connector
e) In a fifth step, filling a body fluid sample tube with blood
f) In a sixth step, removing the body fluid sample tube
g) In a seventh step, removing the device from the patient's arm
h) In an eighth step, cleaning and dressing the wound.
   11. The method of features set 10 further including the repetition of steps (d), (e), (f) to obtain more than one body fluid sample tube filled with a blood sample.

Referring now to FIG. 12A to FIG. 12C, standard lacerators are available on the market. An example of a lacerator that can be used for the process of the present invention is Gentleheel® Heel Incision Device from CardinalHealth, in particular “TGH10X50 Gentleheel®, Toddler, Blue” but other similar incision devices may be used. A lacerator that is appropriate for use in the process of the present invention is able to make a cut in the patient's skin of about 3 mm length for a depth of about 2 mm. Depending on the amount of blood to be collected in a given period of time, the lacerator may have more than one blade to cut more than one wound, making several parallel cuts in one movement. To provide an appropriate wound shape the mechanism 12000 mobilizes the blade 12100 in a combination of a rotation movement and a linear movement. The blade(s) are attached on a lever 12200 that can swivel on a pin 12210, said pin 12210 being free to move within a groove 12400 in the structure of the lacerator. The lever 12200 is actuated via a second lever 12300 that is actuated by the user, optionally with a trigger mechanism as well known in the micromechanics industry, optionally with the help of springs. As a result, before the laceration (FIG. 12A), the blade(s) 12100 are kept out of reach of the user/patient, and a safety feature (not shown) prevents inadvertent actuation of the mechanism 12000 by the user/patient. During the laceration (FIG. 12B), the blade(s) 12100 follow the path defined by the mechanism 12000 and open one or more wound(s) in the user/patient's skin. After the laceration (FIG. 12C), the blade(s) 12100 are stored away of the reach of the user/patient to avoid inadvertent wounding.

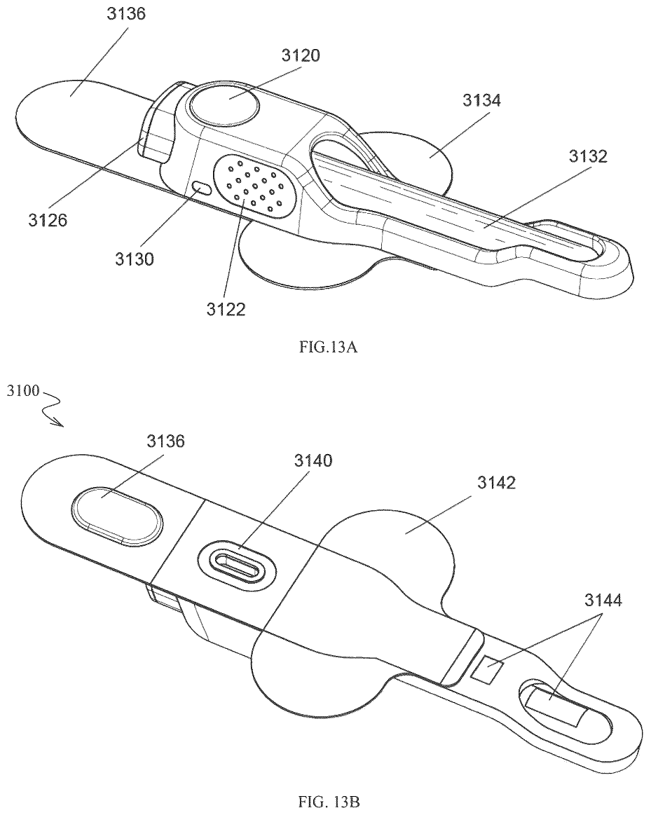

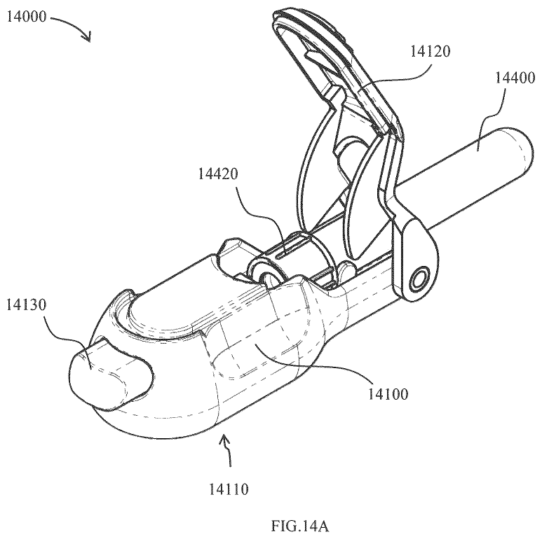

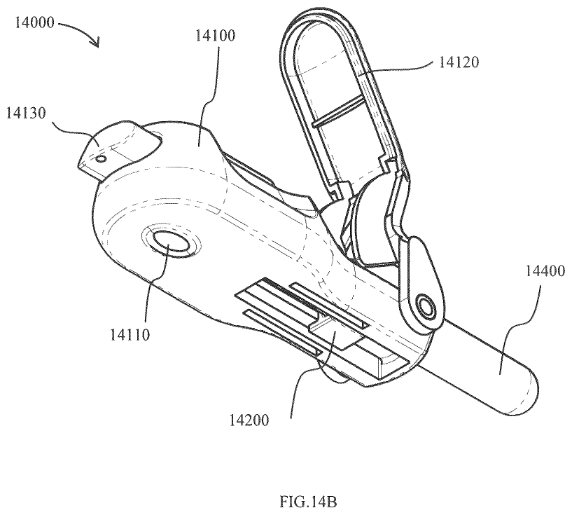

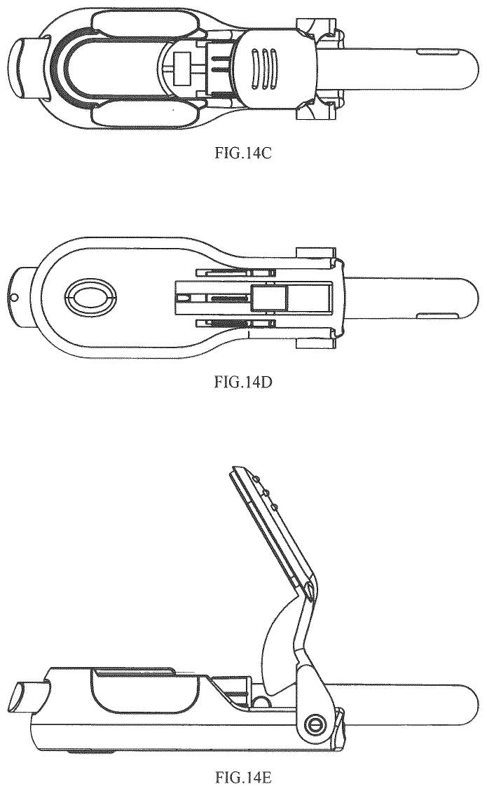

Referring now to FIGS. 13A to 13C The main features of a blood sampling device of the invention are shown. In a first embodiment, the blood sampling device comprises on its top side a grip 3122, a draw blood button 3120, a collect blood button 3126, a flash window 3130 indicating readiness for collecting blood, a blood collection tube 3132, and wing tabs 3134 for device removal as well as dressing 3136 with backer thereon. The blood sampling device 3100 further comprises blood draw reservoir 3140 with wound seal, removal wing tab 3142 with adhesive backing on and machine-readable codes 3144. This first embodiment provides both functionalities of cutting the user/patient's skin and drawing a blood sample. The second embodiment of the device relies on cutting the user/patient's skin with a separate lacerator such as shown in FIG. 12 and the method follows the path described in FIG. 11. Therefore, the second embodiment of the fluid sampling device doesn't have the draw blood button 3120.

Referring now to FIGS. 14A to 14P, the third embodiment of the device 14000 relies on cutting the user/patient's skin with a separate lacerator and the method follows the schematics of FIG. 11, with a lever 14120 at step 11f. In the third embodiment of the fluid sampling device 14000, the device's body 14100 contains a mechanism 14200 actuated by the user/patient via a lever 14120. The mechanism 14200 holds the vacuum tube 14400 by a mechanical feature 14420, for example the mechanical feature is the lid of the vacuum tube 14400. The vacuum tube 14400 carries a unique identification tag 14430 that can be read by humans and/or by machine means. The lever 14120 is equipped with a safety feature 14130 to avoid accidental activation, so the device 14000 is provided with the vacuum tube 14400 already in place in the device and step 11a) of the method of FIG. 11 is not necessary. FIGS. 14A to 14H show the fluid sampling device 14000 before use, so the lever 14120 is in open position. After the user/patient has followed the steps until having created the wound, the user/patient sticks the fluid sampling device 14000 over the wound so that the suction opening 14110 of the fluid sampling device 14000 is placed over the wound. In the case of blood sampling the user/patient uses the mechanical features provided by the adhesive pad for accurate positioning, but in case of other usages such as for collecting puss or venom the user/patient places the fluid sampling device 14000 visually. The user/patient can see that the suction opening 14110 of the fluid sampling device 14000 is filling with fluid by looking through the fluid sampling device's body 14100 that is made of transparent material, or by looking through a transparent window (not shown) provided in the fluid sampling device's body 14100, or by waiting a predefined duration, this duration may last from a few seconds to several minutes, and may be ensured by a timer integrated in the fluid sampling device 14100, by a timer provided by any electronic device and launched by the user/patient, by a timer included in an app running on the user/patient's smartphone monitoring the fluid sampling process. At this point in time the user/patient releases the safety feature 14130 and actuates the lever 14120, mobilizing the mechanism 14200. The mechanism 14200 moves the vacuum tube 14400 towards the fluid sampling device body 14100, impaling the septum 14110 (not shown) of the vacuum tube 14400 on the conduct, connecting the vacuum tube 14000 to the patient's wound, thereby allowing the collected fluid to flow from the wound to the vacuum tube 14400. Alternatively the mechanism 14200 moves the conduct towards the tube 14400 and pierces the septum 14110 (not shown) of the vacuum tube 14400, connecting the vacuum tube 14000 to the patient's wound, thereby allowing the collected fluid to flow from the wound to the vacuum tube 14400. The configuration of the fluid sampling device 14000 is made so that the user/patient pushes on the lever 14120 over the localization of the wound so as to not risk unsticking the fluid sampling device 14000 from the patient's skin. FIGS. 14I to 14P show the fluid sampling device 14000 after the lever 14120 has been pushed.

Referring now to FIGS. 15A to 15B, an adhesive pad 15000 is provided with at least one mechanical feature that allows for the precise placement of the lacerator and of the fluid sampling device. The adhesive pad 15000 includes an adhesive zone 15100 for its attachment to the patient's skin, one or more visual target and mechanical features 15200 allowing the precise positioning of the lacerator and of the fluid sampling device, and a dressing part 15300 for covering the wound after the fluid sampling process. The target and mechanical feature(s) 15200 may have the shape of a ridge against which the user/patient can push the lacerator and the fluid sampling device. Before the fluid sampling process starts, the adhesive zone 15100 is covered with backers 15110, the target and mechanical positioning feature(s) 15200 is covered with backers 15210 and the dressing part 15300 with backer 15310. The user/patient removes the respective backers as appropriate in order to follow the fluid sampling process.

Referring now to FIGS. 16A to 16F, some key steps of the method using the adhesive pad 16100, a lacerator and the fluid sampling device 16200 of the invention to sample blood on the arm 16300 of a patient are shown.

FIG. 16A illustrates the step of installing the adhesive pad 16100 on the arm 16300 of the patient and exposing the target and mechanical feature 16110.

FIG. 16B illustrates the step of installing the fluid sampling device 16200 using the target and mechanical feature 16110 over the wound 16310 after it has been cut open with the lacerator.

FIG. 16C illustrates the step of activating the sampling with the fluid sampling device 16200 by pushing the safety button 16230 and then closing the lever 16240.

FIG. 16D illustrates the step of waiting and monitoring the fluid 16400 flowing into the vacuum tube 16250 of the fluid sampling device.

FIG. 16E illustrates the step of removing the fluid sampling device 16200 after sampling.

FIG. 16F illustrates the step of dressing the wound 16310 with the adhesive pad 16100.

Referring now to FIG. 17, the geometry of the suction chamber 17100 of the fluid sampling device 17000 of the invention is adapted to provide an airtight interface at the suction opening 17200 to the patient's skin and to allow the collecting and free flowing of the sampled fluid through the conduct 17300 into the vacuum tube. The internal volume of the suction chamber 17100 is as small as possible to avoid unnecessary losses of vacuum and stagnation of the sampled fluid. The internal surface 17110 of the suction chamber 17100 and the internal surface 17310 of the conduct 17300 are made as slick and flush as possible, optionally coated with an antiadhesive coating, optionally coated with an anticoagulant coating, to minimize the risk of coagulation of the sampled fluid and to promote an as free as possible flow of the sampled fluid through the suction chamber 17100, through the conduct 17300 and into the vacuum tube. The edge 17210 of the suction opening 17200 is made with a concave shape, so as to avoid any of the sampled fluid to stagnate at the suction opening 17200 and/or to slip below the sampling device 17000. The top side 17120 and the bottom side 17130 of the suction chamber 17100 form together a funnel leading from the suction opening 17200 to the conduct 17300. The bottom side 17130 of the suction chamber 17100 is made so as to present a downwards slope relative to gravity when the device 17000 is placed on the arm of the patient.

Referring now to FIGS. 18A to 18P, a fourth embodiment of the method of using the system of the invention is shown, and the specific features of the user authentication system 1900, of the device authentication system 1902, of the capillary blood sampling device 3100, 4100 and of the adhesive integrated dressing 3174 included in the capillary blood sampling device 3100, 4100 can be deducted.

FIG. 18A shows step 1 of the fourth embodiment of the method of using the system of the invention. Step 1 comprises: a) loading a dedicated App for use with the invention via scan website or prescription; b) executing a consent form; c) entering demographics information; d) photographing government issued ID or another identification means; and e) taking a selfie for association with the patient and the device as well as the sample taken.

FIG. 18B shows step 2 of the fourth embodiment of the method of using the system of the invention. Step 2 comprises: a) opening package; b) reading instructions; c) removing contents from package; d) scanning QR code on kit box to launch App and guide; e) checking contents; and f) cleaning phone with sanitary wipe included in kit.

FIG. 18C shows step 3 of the fourth embodiment of the method of using the system of the invention. The step 3 comprises: a) deploying integrated phone stand in box; b) wiping down phone with included sanitary towelette; and c) washing hands.

FIG. 18D shows the step 4 of the fourth embodiment of the method of using the system of the invention. Step 4 comprises: a) taking a selfie with phone on stand; b) starting video of self with face and arm in the frame.

FIG. 18E shows the step 5 of the fourth embodiment of the method of using the system of the invention. Step 5 comprises: a) following App guide (reading text/reviewing figures); b) preparing arm with a warm towel; c) opening alcohol towelette from kit; and d) wiping site on upper arm with alcohol, then letting dry.

FIG. 18F shows step 6 of the fourth embodiment of the method of using the system of the invention. step 6 comprises: a) removing device from kit packaging; and b) presenting QR code on device and tube to camera on stand.

FIG. 18G shows step 7 of the fourth embodiment of the method of using the system of the invention. Step 7 comprises: a) removing adhesive backing; and b) adhering device to upper arm.

FIG. 18H shows step 8 of the fourth embodiment of the method of using the system of the invention. Step 8 comprises: confirming video of self with face device are in the frame.

FIG. 181 shows the step 9 of the fourth embodiment of the method of using the system of the invention. Step 9 comprises: a) pushing a first button; b) waiting until in flash window an indication is indicated; c) confirm the indication; and d) if no indication is indicated in prescribed time, follow guide to return kit.

FIG. 18J shows the step 10 of the fourth embodiment of the method of using the system of the invention. Step 10 comprises: a) pushing a second button; and b) waiting for timer on App to indicate tube is full with a means of communication progression.

FIG. 18K shows the step 11 of the fourth embodiment of the method of using the system of the invention. Step 11 comprises: a) pulling on wing tabs to peel the device off the arm; b) leaving dressing on arm; and c) returning device to kit box.

FIG. 18L shows the step 12 of the fourth embodiment of the method of using the system of the invention. Step 12 comprises: a) removing dressing backer revealing gauze pad and adhesive; and b) folding gauze dressing down over wound.

FIG. 18M shows the step 13 of the fourth embodiment of the method of using the system of the invention. Step 13 comprises: a) popping out end of vial from device; and b) pulling vial out of device.

FIG. 18N shows the step 14 of the fourth embodiment of the method of using the system of the invention. Step 14 comprises: a) securing sample vial in biohazard pouch from kit; and b) securing device in separate biohazard pouch from kit.

FIG. 18O shows the step 15 of the fourth embodiment of the method of using the system of the invention. Step 15 comprises: a) stopping video; b) removing phone from kit stand; c) collapsing phone stand; and d) securing both biohazard pouches in kit box.

FIG. 18P shows the step 16 of the fourth embodiment of the method of using the system of the invention. Step 16 comprises: a) sealing kit box in return shipping pouch; b) scanning QR code on shipment package; c) mailing package; d) confirming shipment; e) App notifies patient with test results.

Referring now to FIGS. 19A to 19D, more details about the specific features of the adhesive integrated dressing 3174 included in the capillary blood sampling device 3100 of the invention are shown. The blood sampling device 3100 comprises an adhesive 3150 around wound site separate from the adhesive 3152 on the bottom of the device. The blood sampling device further comprises an adhesive backing 3154 which is removably located on the adhesive 3150, 3152, the adhesive backing 3154 is removed prior to the device being applied to a patient's body. Once the device is placed on a patient's arm, the device may be pulled up 3157 on a wing tab 3156 to peel the device off of the patient's skin after tube 3132 is filled. The adhesive 3150 surrounding the wound site 3162 remains behind on skin with an encompassing features to restrain the blood from flowing out of the exposed site area temporarily. Then, a dressing backer 3164 is peeled off 3165 so as to reveal gauze pad 3166 surrounded by adhesive 3170. The dressing 3174 comprise a dressing tab 3172 without adhesive. The dressing tab 3172 is pullable 3173 to fold the gauze pad onto the wound site and may be affixed to cover the wound.

As a result, the adhesive integrated dressing 3174 is adapted to ensure the attachment of the blood sampling device 3100 to the patient's skin, the air-tightness between the patient's skin and the blood sampling device 3100 during the sampling process, and the dressing of the wound after the sampling process.

Capillary blood sampling devices for non-medically trained users of the current art usually create the wound in the patient's skin by perforation with one or more needles, or even without perforation, which only allows for the collection of relatively small volumes of fluid, typically less than 150 μl in 5-10 minutes.

In another embodiment, the present invention provides means for the capillary blood sampling device 10, 1100, 2010, 2210, 3100, 4100 to make a significantly larger cut than what is usually known in existing capillary blood sampling devices, so that a significantly larger amount of blood, typically more than 500 μl, preferably 1 ml can be collected over a reasonable period of time, typically less than 15 minutes, preferably less than 10 minutes. The capillary blood sampling device of the invention uses one or more cutting blade(s) 1004 (e.g., but not limited to cutting blades 302, 5450, 5456, 3260, 3360, 3460), instead of one or more needle(s), so that the user/patient's skin is lacerated instead of punctured. For the purpose of the present disclosure, the functioning of the sampling device of the invention is described assuming it contains one cutting blade, but it must be understood that the sampling device of the invention may contain more than one cutting blade in order to increase the amount of blood collected and/or to decrease the blood collection time. Moreover, the current invention provides cutting solutions that favor a quick healing of the wound after the blood sampling is complete. The purpose of the invention is therefore to create a wound in the user/patient's skin that has an optimal depth for cutting as many capillaries as possible, while avoiding unnecessary wound width and length so that the natural healing of the wound can happen as fast as possible after the blood collection. The ideal cutting depth may vary as function of the patient's age, gender, ethnical group and/or health condition, as a result several adapted versions of sampling devices may be provided. Typically ideal cutting depth is between 1 mm and 2 mm.

Referring now to FIGS. 20A to 20C, in a fourth embodiment the cutting blade 3260 of a device of the invention is made of a single part, typically out of sheet metal or spring steel, but may be also made of other materials, including composite materials presenting the appropriate mechanical properties. The cutting blade 3260 has an end 3261 facing the sampling device structure so that it can be attached to it, followed by an elastic zone 3262 which is bent elastically when the cutting blade is ready to be used. In this way, the cutting blade 3260 contains all the energy necessary for its movement, and the user/patient only needs to release it by depressing the draw blood button 3120. The elastic zone 3262 has an appropriate, preferably flat cross-section so as to provide a preferred release trajectory perpendicular to its attachment end 3261. In one embodiment, after the elastic zone 3262, the cutting blade is twisted by 90° in the area 3263 so as to provide a blade section 3264 which is in the same plane as the release trajectory. The blade section 3264 has a high rigidity in the direction of the release trajectory.

The elastic zone may take the form of a multiturn torsion spring, such as can be found in clothes pins, in such case the cross-section of the blade is round and the varying stiffnesses necessary for the function are obtained by variation of the thermal treatments of the different zones of the blade. Towards the end of the blade section 3264, a cutting edge 3265 is provided so as the lacerate the user/patient's skin 3290. After completion of the movement of the cutting, edge 3265 faces away from the user/patient's reach. Optionally the cutting blade 3260 includes a finger 3270 that interacts with the device's structure elements 3242, 3244, 3246 in order to bias the natural release trajectory 3250 of the cutting blade 3260 when released to obtain a modified trajectory 3240.

Typically, the natural release trajectory 3250 of the cutting blade 3260 is substantially circular, elliptical or spiral. As a result, the laceration in the patient's skin 3290 is substantially circular and with a relatively large radius, and the wound length 3296 is relatively long for a small portion at the desired depth 3292. When elements of the device's structure 3242, 3244, 3246 interact with the finger 3270 of the cutting blade 3260, the resulting trajectory 3240 of the cutting blade 3260 is modified so that the resulting laceration of the patient's skin 3290 has a steeper dive and retraction path, resulting in a shorter wound length 3294 for a longer proportion of the wound at the desired depth 3292. The modified trajectory 3240 allows for a larger volume of capillary blood to be collected, for a globally smaller wound, favoring a quicker healing of the wound after the blood collection.

Referring now to FIG. 20B, as an example, the element of the device's structure 3242 is made to locally extend the radius of natural release trajectory 3250, and the element of the device's structure 3244 is made to locally shrink the radius of the natural release trajectory 3250.

Referring now to FIG. 20C, a further example the element of the device's structure 3246 is made to locally shrink the radius of natural release trajectory 3250.

Referring now to FIGS. 21A to 21B, in a fifth embodiment the cutting blade 3360 of a device of the invention is made of a single part, typically out of sheet metal, but may be also made of other materials, including composite materials presenting the appropriate mechanical properties. The cutting blade 3360 has an end 3361 facing the sampling device structure so that it can be attached to it, followed by an elastic zone 3362 which is twisted elastically when the cutting blade 3360 is ready to be used. In this way, the cutting blade 3360 contains all the energy necessary for its movement, and the user/patient only needs to release it by depressing the draw blood button 3120. The elastic zone 3362 has an appropriate, preferably flat cross-section so as to provide a preferred, planar natural release trajectory 3350. In one embodiment, after the elastic zone 3362, the cutting blade is twisted by 90° in the area 3363 so as to provide a blade section 3364 which is in the same plane as the release trajectory. The blade section 3364 has a high rigidity in the direction of the release trajectory. Towards the end of the blade section 3364, a cutting edge 3365 is provided so as to lacerate the user/patient's skin (not represented). After completion of the movement, the cutting edge 3365 faces away from the user/patient's reach. Optionally, the cutting blade 3360 includes a finger 3370 that interacts with the device's structure element 3342, in order to bias the natural release trajectory 3350 of the cutting blade 3360 when released, generating a modified release trajectory 3340.

Typically the natural release trajectory 3350 of the cutting blade 3360 is substantially circular or spiral. As a result, the laceration in the patient's skin is substantially circular and with a relatively large radius, and the wound length is relatively long for a relatively small portion at the desired depth. When an element of the device's structure 3342 interacts with the finger 3370, the resulting trajectory 3340 of the cutting blade 3360 can be modified so that the resulting laceration of the patient's skin has a steeper dive and retraction path, resulting in a shorter wound length, and a longer proportion of the wound at the desired depth. The modified trajectory 3340 allows for a larger amount of blood volume to be collected, for a generally smaller wound, favoring a quicker healing of the wound after the blood collection. As an example, the element of the device's structure 3342 is made to shrink locally the radius 3352 of the natural release trajectory 3350 by an offset 3341 on a part of the release trajectory 3340. In addition, the element of the device's structure 3342 can be made with a more complex shape so as to provide a more elaborated modified trajectory 3340.

Referring now to FIGS. 22A to 22C, in a fourth embodiment the cutting blade 3460 of a device of the invention is made of a single part, typically out of sheet metal, but may be also made of other materials, including composite materials presenting the appropriate mechanical properties. Referring now to FIG. 22A, the cutting blade 3460 has a rotative attachment 3466 to the device's structure and a cutting edge 3465, which is kept in retracted position prior to the cutting process.

Referring now to FIG. 22B, when actuated the cutting blade 3460 engages into the patient's skin 3490 in a rotative movement, but the rotative movement is limited by the structure so that the patient's skin is not completely lacerated.

Referring now to FIG. 22C, after the laceration the cutting blade 3460 is retracted in reverse rotative movement, leaving a wound under the patient's skin that is much smaller than if the laceration had been made completely. As a result, a larger amount of blood volume can be collected thanks to the deep laceration, but at the surface of the patient's skin 490, the opening is smaller, favoring a quicker healing of the wound after the blood collection.

Referring now to FIGS. 23A to 23D, in a fifth embodiment the cutting blade 3660 of a device of the invention may be a standard scalpel blade or any other rigid blade, typically out of sheet metal, but may be also made of other materials, including composite materials presenting the appropriate mechanical properties. The cutting blade 3660 has a cutting edge 3665, positioned substantially parallel to the skin of the user/patient 3690. The cutting blade is guided in a linear movement by the device's mechanism (not shown) in order to penetrate the user/patient's skin at a substantially non-orthogonal angle (FIG. 23B). In this way, the cutting edge 3665 penetrates completely in the skin of the user/patient, reaching a substantially uniform depth along all its length, creating a substantially rectangular wound, oriented non-orthogonally in the user/patient's skin (FIG. 23C). When the cutting blade is retracted (FIG. 23D), the flap generated by the non-orthogonal wound closes naturally the wound's entrance, favoring a quicker healing of the wound after the blood collection.

In another embodiment, the invention provides for verification of self-administered medical processes. An objective of this embodiment is to make sure the user/patient is identified and to make sure the blood in the sampling tube(s) is the blood of this patient

What may be at stake here is:
Reliable blood sampling process;
Authorization to work, to travel, to be in contact with family in times of epidemics;
Authorization to use a certain treatment;
Automatized detection of epidemics by large-scale sampling programs;
Verification of the efficiency of a given treatment (payment subject to treatment success);

Still further, payment of a treatment may be at stake, contingent on vaccination and only if fully administered. Therefore, making a secure patient & process verification is crucial.

In another aspect, verification of self-administered medical processes is important. For example, use of a smartphone application may be made in order to make a video (or a time-lapse) where the patient's face as well as the process itself are visible in the video all along the process duration. A comparison of the patient's ID with the patient's face may be made. The patient's face recognition and whole process may be observed. The system could be configured to launch the process only when all ID's are confirmed. Automatized handling of the logistics (sampling tube collection & transport, treatment re-supply, etc.) may be implemented. In the case that the smartphone app is configured to analyze the video in real-time, step-by-step instructions may be provided to the patient in real-time, while the process is being executed. Other features could be included such as automated monitoring of process performance. Despite the potential, there will always be challenges to deal with, particularly in degraded conditions, when dealing with insufficient network coverage at the moment of running the process. Optionally, temporary network coverage may be provided via drones/balloons for the duration of the treatment campaign. It would be best however, if the App is able to run independently of network coverage. Saving all data on the smartphone and making verifications at a later stage may also be desirable. Respecting private data regulations is an issue of course. The App may be configured to run a first analysis and then an encryption routine to make private data unreadable, with full video optionally saved for later use in case of need (e.g. formal proof in a court).

Main components of the system include a smartphone or laptop/computer or similar and camera (may be included in the smartphone/laptop/computer), the application to be run by the smartphone or laptop/computer; the device for running the injection/sampling process, which may be re-usable. In addition, the treatment to be injected/one or more empty tubes/vials to contain the collected blood sample(s) (may be under vacuum).

As for some key features of the system of the invention, the app must be able to read treatment container/sampling tube's unique ID using for example a standard bar-code/QR-code. The App must be able to identify key process steps. The device may need to include automatized wireless signal emission (information: ID/process started/process ongoing/process finished/error). Such signal may be visible (e.g. blinking/colored LEDs) for easy interpretation in the video. The device of the invention may optionally include visible features/landmarks for easier orientation verification in the video. The App should be able to launch the treatment process. The device is optionally equipped with remote triggering feature and advantageously includes a unique ID.

Referring now to FIGS. 24A to 24C, visible features 2600 integrated in the device 2610 of the invention that can be recognized easily may optionally take the form of the combination of different high-contrast patterns 2620, 2630 applied on a mobile part (in this example a button 2650) and visible through a window 2612 only one at a time. As a result, predefined positions of the mobile part (button 2650 in high position in FIG. 24B, button 2650 in low position in FIG. 24C) can be easily recognized by the human observer, or easily identified by an image analysis software. Colored parts and/or features may also protrude and/or disappear from/in the device at specific steps of the process so as to materialize the completion of the step and be easily recognized by the human observer, or easily identified by an image analysis software.

For the purpose of the present disclosure the words blade, cutting blade or lancet are used indifferently and must be understood as equivalents.

The invention may have other uses. For example, it may be applied to another medical treatment other than blood sampling (the device may be an injection device, a pills distributor). It may be applied to filling a voting form at home, signing documents, proving one's ID during a teleconference, or taking a remote exam.

Note that in this application, where ever blood sampling is mentioned, it should be understood that puss or venom can be substituted for blood.

The invention can be summarized as including the following feature sets:

1. A disposable fluid sampling device including:
(a) body fluid sampling means, optionally body fluid sampling means for auto-sampling;
(b) optionally, analysis means configured to use one or more droplet(s) of the sampled fluid to analyze the fluid; and
(c) an interface for a medical analysis tube meeting size and interface standards, the tube adapted to be filled with a sample of fluid for analysis in a point of care or medical lab, wherein the device includes an interface for and a vacuum tube, the vacuum tube adapted to provide suction necessary to fill the vacuum tube with the fluid.
   2. A method of using the fluid sampling device of feature set 1, wherein, in a first step, a test subject is tested for a pathogen, and, if tested positive, in a second step, a treatment and/or quarantine protocol is initiated to ensure that a test subject having a positive test is handled in a manner which helps minimize the spread of the pathogen.
   3. A lancet adapted for making a laceration in the skin of a user/patient for a disposable fluid sampling device of feature set 1 used for collecting a body fluid, wherein the lancet construction is made in one piece of material and is disposed in a holder so as to enable its providing of the energy for movement and the guiding of its movement.
   4. The sampling device of feature set 1, wherein a fluid extraction passage or conduit from the patient's wound to the vacuum tube is formed in a needle.
   5. The sampling device of feature set 1, wherein the user is a non-medically trained user.
   6. The sampling device of feature set 1, wherein the fluid reservoir is the vacuum reservoir.
   7. The sampling device of feature set 1, wherein the fluid is, at least in part, blood.
   8. The sampling device of feature set 1, wherein the fluid is, at least in part, puss.
   9. The sampling device of feature set 1, wherein the fluid is, at least in part, venom.
   10. The sampling device of feature set 1, wherein, optionally, the device is adapted to use one or more droplet(s) of the sampled body fluid, to analyze the body fluid.
   11. The sampling device of feature set 1, wherein the device includes a vacuum tube and an interface therefor, the vacuum tube providing suction necessary to fill the vacuum tube with the body fluid.
   12. The sampling device of one of feature sets 1, 4 to 11, wherein the medical analysis tube is a standard medical analysis tube.
   13. A method of capillary blood sampling, optionally self sampling, the method includes at least the steps of:
a) placing an adhesive pad at the intended location for the capillary blood sampling, the adhesive pad including at least one mechanical localization feature and optionally visual indications;
b) lacerating the user/patient's skin with a lacerator, thereby opening at least one wound in the user/patient's skin, the lacerator being positioned using the at least one mechanical localization feature of the adhesive pad;
c) collecting the patient's capillary blood with a fluid sampling device, the device being positioned on the at least one wound using the at least one mechanical localization feature of the adhesive pad and the device using vacuum to collect blood from the at least one wound and to fill a sample analysis tube; and
d) dressing the at least one wound using a foldable part of the adhesive pad.
   14. The device of feature set 1, wherein the device further includes an adhesive pad, the adhesive pad adapted for application at the intended location for the capillary blood sampling, the adhesive pad including at least one mechanical localization feature and optionally visual indications, the adhesive pad further containing at least one mechanical feature that allows for the precise placement of the lacerator and of the fluid sampling device and a foldable portion adapted for covering the at least one wound after the blood collection.
   15. The device of feature set 1 or 14, wherein the device further includes a lacerator including a lancet adapted for making a one or more lacerations in the skin of a user/patient, wherein the lancet construction is made in one piece of material and provides the energy and the guiding for its movement.
   16. The device of feature set 1, 14 or 15, wherein the device further includes a lacerator including a lancet adapted for making a one or more lacerations in the skin of a user/patient, wherein the lacerator contains one blade for each of the one or more lacerations and makes the one or more lacerations simultaneously.
   17. The lacerator of feature set 16, wherein when lacerating the patient's skin the trajectory of the at least one blades follows a trajectory that combines at least one rotary and at least one linear component of movement.

Further, the invention should be considered as comprising all possible combinations of every feature described in the instant specification, appended claims, and/or drawing figures which may be considered new, inventive and industrially applicable.

It should be appreciated that the particular implementations shown and herein described are representative of the invention and its best mode and are not intended to limit the scope of the present invention in any way.

As will be appreciated by skilled artisans, the present invention may be embodied as a system, a device, or a method.

Moreover, the system contemplates the use, sale and/or distribution of any goods, services or information having similar functionality described herein.

The specification and figures should be considered in an illustrative manner, rather than a restrictive one and all modifications described herein are intended to be included within the scope of the invention claimed. Accordingly, the scope of the invention should be determined by the appended claims (as they currently exist or as later amended or added, and their legal equivalents) rather than by merely the examples described above. Steps recited in any method or process claims, unless otherwise expressly stated, may be executed in any order and are not limited to the specific order presented in any claim. Further, the elements and/or components recited in apparatus claims may be assembled or otherwise functionally configured in a variety of permutations to produce substantially the same result as the present invention. Consequently, the invention should not be interpreted as being limited to the specific configuration recited in the claims.

Benefits, other advantages and solutions mentioned herein are not to be construed as critical, required or essential features or components of any or all the claims.

As used herein, the terms “comprises”, “comprising”, or variations thereof, are intended to refer to a non-exclusive listing of elements, such that any apparatus, process, method, article, or composition of the invention that comprises a list of elements, that does not include only those elements recited, but may also include other elements described in the instant specification. Unless otherwise explicitly stated, the use of the term “consisting” or “consisting of” or “consisting essentially of” is not intended to limit the scope of the invention to the enumerated elements named thereafter, unless otherwise indicated. Other combinations and/or modifications of the above-described elements, materials or structures used in the practice of the present invention may be varied or adapted by the skilled artisan to other designs without departing from the general principles of the invention.

The patents and articles mentioned above are hereby incorporated by reference herein, unless otherwise noted, to the extent that the same are not inconsistent with this disclosure.

Other characteristics and modes of execution of the invention are described in the appended claims.

Further, the invention should be considered as comprising all possible combinations of every feature described in the instant specification, appended claims, and/or drawing figures which may be considered new, inventive and industrially applicable.

Additional features and functionality of the invention are described in the claims appended hereto. Such claims are hereby incorporated in their entirety by reference thereto in this specification and should be considered as part of the application as filed.

Multiple variations and modifications are possible in the embodiments of the invention described here. Although certain illustrative embodiments of the invention have been shown and described here, a wide range of changes, modifications, and substitutions is contemplated in the foregoing disclosure. While the above description contains many specific details, these should not be construed as limitations on the scope of the invention, but rather exemplify one or another preferred embodiment thereof. In some instances, some features of the present invention may be employed without a corresponding use of the other features. Accordingly, it is appropriate that the foregoing description be construed broadly and understood as being illustrative only, the spirit and scope of the invention being limited only by the claims which ultimately issue in this application.

What is claimed is:

1. A fluid sampling device (10) adapted to take a sample of a body fluid (50), preferably one or more droplets of the body fluid (50) for optional immediate analyses and quantities of body fluid similar to those collected by phlebotomy for further analyses at a point of care or in a medical lab, the disposable fluid sampling device optionally adapted for auto-sampling; (and optionally), wherein the disposable fluid sampling device (10) includes a vacuum interface for interfacing a vacuum source adapted for providing suction necessary to fill the at least one receptacle (20) with the body fluid (50).

2. The fluid sampling device (10) of claim 1, whereby the vacuum source is formed as a receptacle (20) for accommodating body fluid (50).

3. The fluid sampling device (10) of claim 1, whereby the vacuum source is different from the receptacle (20) for accommodating the body fluid (50).

4. The fluid sampling device (10) of claim 3, including a connector (114) allowing more than one receptacle (20, 120) to be connected in a sequence to the device (10) so as to fill more than one receptacle (20, 120) with a body fluid sample.

5. The fluid sampling device (10) of claim 3, whereby the vacuum source is a vacuum tube.

6. The fluid sampling device (10) of claim 5, including a connector (118) allowing more than one vacuum tube to be connected in a sequence to the device (10) so as to fill more than one receptacle (20, 120) with a body fluid sample.

7. The fluid sampling device (10) of claim 1, including a tube interface, the tube interface adapted to interface at least one receptacle, the receptacle preferably being a sample receptacle or a medical analysis receptacle (20),

8. The fluid sampling device (10) of claim 7, the first vacuum source (20, 60) is interconnected to a first of the receptacles (114, 118), and the second vacuum source (60, 20) is interconnected to a second of the receptacles (118, 114).

9. The fluid sampling device (10) of claim 2, including an analysis arrangement optionally connectable to the tube interface, whereby the analysis arrangement is adapted to analyze the body fluid (50).

10. The fluid sampling device (10) of claim 8, comprising a valve (40), preferably a stopcock for changing the inlet from the at least first to an at least second body fluid reservoir (34, 36), each of the body fluid reservoirs being formed of said vacuum source or said receptacle.

11. The fluid sampling device (10) of claim 1, including a watertight fluid extraction passage or conduit for connecting a patient's wound (100) and the receptacle (20), the watertight fluid extraction passage or conduit respectively comprises a flexible tube (16).

12. The fluid sampling device (10) of claim 1 comprising a heating element (22) which is attachable to the skin in the vicinity of an area, wherein the body fluid (50) is drawn from the body.

13. The fluid sampling device (10) of claim 11, wherein the heating element (22) is thin enough to be located between the receptacle and the patient's skin during the blood collection process, preferably between 0.5 mm and 4 mm, more preferably between 1 mm and 3 mm thick.

14. The fluid sampling device (10) of claim 1, where the receptacle for accommodating the body fluid (50) is made from translucent material, the translucent material preferably being colored.

15. The fluid sampling device (10) of claim 1, wherein the fluid sampling device (10) includes a connector (114) connected to the device (10) via a flexible tube (112) to receive the blood collection/sample receptacle (120), and a conduit (116) to provide vacuum to the blood collection/sample receptacle (120) and to the device (10).

16. The fluid sampling device (10) of claim 15, wherein the fluid sampling device (10) includes a second connector (118) connected to the receptacle via a conduit (116) to connect to a vacuum tube.

17. A method for sampling body fluid, optionally self sampling, the method including the following steps:
a) in a first step, installing the fluid sampling device (10) according to claim 1 on the patient's arm, the fluid sampling device (10) including a first vacuum source insertable into a vacuum interface;
b) in a second step, triggering the skin laceration for lacerating the skin;
c) in a third step, inserting a first vacuum source into the vacuum interface;
d) in a fourth step, filling a receptacle (20) of the fluid sampling device (10) with a body fluid (50); and
e) in a fifth step, removing the first vacuum source.

18. The method of claim 17, further including a repetition of steps (c), (d), (e) executed for activating a second vacuum source of the fluid sampling device (10) and optionally to fill a second receptacle with a sample of the body fluid (50).

19. The method of claim 17, wherein the fluid sampling device (10) includes a connector (114) connected to the device (10) via a flexible tube (112) to receive the blood collection/sample receptacle, and a conduit (116) to provide vacuum to the blood collection/sample receptacle (120) and to the device (10).

20. The method of claim 19, wherein the fluid sampling device (10) includes a a second connector (118) connected to the receptacle via a conduit (116) to connect to a vacuum tube.

21. The method of claim 12 or 13, where a heating element (22) of the fluid sampling device (10) is, and preferably remains, activated for a period of time, to keep the patient's skin at an elevated temperature in order to increase body fluid draw during the sampling process.

22. A receptacle (80) to be used with the fluid sampling device (10) of claim 1, where a tamper evident label (89) is connectable to a cap (88) of the receptacle (80), so that be only opened in an non-tamper-evident manner at the lab performing the body fluid sample analysis, thereby allowing confirmation of the body fluid sample origin.

23. The fluid sampling device (10) of claim 1 wherein an extra vacuum source (60) is used to collect the body fluid (50) by providing vacuum to a non-vented first tube (20).

24. The fluid sampling device (10) of claim 1, wherein an amount of additives (84, 86) present in the receptacles (20, 80) for capillary sampling is adapted to the amount of body fluid (50) to be collected. (typically 1 to 1.5 ml).

25. The fluid sampling device (10) of claim 1, wherein the level of vacuum in the vacuum source (20, 60) is adapted to the need of capillary sampling, and so may differ from Annex B of a norm of ISO-6710.

26. The method of capillary blood sampling of claim 25, wherein laceration of the skin for blood sampling is made in a direction parallel to the receptacle, or parallel to the main length of the conduit (16) or main fluidic passage leading the body fluid to the receptacle.
